(19) United States
(12) Patent Application Publication
(10) Pub. No.: US 2010/0221809 A1
(43) Pub. Date: Sep. 2, 2010

(54) Compositions and Methods for the Isolation of Biologically Active Proteins

(75) Inventors: Henry J. Forman, Merced, CA (US); Matthew P. Meyer, Merced, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 12/638,806

(22) Filed: Dec. 15, 2009

Related U.S. Application Data
(60) Provisional application No. 61/140,550, filed on Dec. 23, 2008.

(51) Int. Cl. C12N 9/20; C07C 271/42; C07C 327/02; C07F 9/02; C07C 381/00

(52) U.S. Cl. 435/198; 560/115; 564/74; 568/14; 560/147

(57) ABSTRACT

This invention is directed to compositions and methods for the isolation and further characterization of eukaryotic, mammalian, and/or human PC-PLC. In addition, the present invention discloses methods for the synthesis of an affinity chromatography resin for the isolation and purification of biologically active proteins, including eukaryotic PC-PLC.

### CROSS-REFERENCE TO RELATED APPLICATIONS

This application claims the benefit under 35 U.S.C. §119(e) of U.S. Provisional Application Ser. No. 61/140,550, filed Dec. 23, 2008, the contents of which are incorporated by reference in its entirety.

### BACKGROUND OF THE INVENTION

Phosphatidylcholine-specific phospholipase C (also known as phosphotidylcholine cholinephosphohydrolase, or lecithinase C, termed PC-PLC (classification EC 3.1.4.3)) is an enzyme found in a varied of microorganisms, yeast and mammals. In microbes it has been isolated from various species such as Clostridium perfingens, Clsotridium novyi, Bacillus cereus, Bacillus thuringeiensis, Pseudomonas aeruginosa, Listeria moncytogenes, Helicobacter pylori and Staphylococcus aureus. U.S. Pat. No. 6,660,494, col. 1, lines 23-23, citing J. G. Songer (1997) Trends in Microbiology 5:156; Baine (1988) J. Gen. Micro. 134:489 and A. Coffey et al. (1996) Applied and Environ. Micro. 62:1252.

The natural substrate for PC-PLC is phosphatidylcholine (PC). Phospholipids are the primary structural constituents of biological membranes. The products of PC-PLC action on PC are diacylglycerol (DAG), which is a second messenger that activates protein kinase C and some other enzymes, and phosphocholine, which does not have a known signaling role.

Phospholipases are classified by their site of action on the phospholipid molecule. PLA-1 hydrolyzes the bond between the fatty acid and the glycerine residue at the 1-position of the phospholipid. PLA2 hydrolyzes the 2-acyl or the central acyl group. PLC and phospholipase D (PLD) cleave on different sides of the phosphodiester linkage of the phospholipid.

PLC-α belongs to a family of enzymes known as disulfide isomerases that are important in transmembrane signal transduction and has little or no actual phospholipase activity. Paragraph [0007] of U.S. Patent Publ. No. 2005/0026235A1. Activated PLC catalyzes the hydrolysis of phosphatidylinositol 1,4,5-triphosphate (IP3). Several distinct isoforms of PLC have been identified and characterized, PLC-β, PLC-γ and PLC-δ. The three isoenzymes have two regions of significant homology: the first region (designated X) contains about 170 amino acids and the second contain (designated Y) contains about 260 amino acids. Id.

Methods are known for the detection of PC-PLC activity and have been reviewed in I. L. Krug and C. Kent (1981) Methods in Enzymology 72:347. See U.S. Pat. No. 6,660,494, col. 1, lines 46-50. One common method detects phosphocholine produced by the enzyme from the natural substrate phosphatidylcholine. Molecular Probes (Eugene, Oreg.) also sells an assay for phospholipase activity. The assay utilizes a fluorogenic probe for hydrogen peroxide. These earlier methods only allow the amount of enzyme present in the sample at the time the sample was taken. U.S. Pat. No. 6,660,494, col. 1 at lines 46-55. Kurioka et la. (1976) Analytical Biochemistry 75:281.

U.S. Pat. No. 6,660,494 discloses compositions and methods to detect PC-PLC using chromogenic substrates containing a core 3-indoxyl choline phosphate compound. U.S. Patent Publ. No. 2005/0026235A1 discloses a method to detect phospholipase which utilizes phosphate-containing lipid complex that comprises a labeled substrate and a fluorescent label attached to the phosphate.

Two groups have used antibodies made to the Bacillus cereus PC-PLC (Egan, B. S. et al. J. Biol. Chem. (1999) 274:9098-9107 and Meng, A. et al. (2004) Exp. Cell Res. 292:385-392) to study mammalian PC-PLC. Nevertheless, the characteristics of mammalian PC-PLC remain clouded by the lack of identification of the protein sequence or the gene. The entire database of non-redundant gene sequences in all eukaryotic genome databases using several search techniques (BLASTP, PSI-BLAST) on the NCBI web site failed to identify a match with the Bacillus cereus PC-PLC sequence. While PC-PLC and proteins from other bacteria were found with high homology, no significant match to any eukaryotic protein was found. Any value of E>0.0001 is considered insignificant. The best value in any mammalian database was E=0.023 for a very short protein fragment (42 aa) from a mixed-lineage leukemia translocation. This raises questions about the degree of homology between bacterial and mammalian PC-PLC and the validity of protein purification using antibodies to bacterial PC-PLC ((Egan, B. S. et al. J. Biol. Chem. (1999) 274:9098-9107 and Meng, A. et al. (2004) Exp. Cell Res. 292:385-392).

Thus, a need exist to provide a method to purify and characterize eukaryotic PC-PLC. This invention provides composition and methods useful for the isolation and further characterization of eukaryotic mammalian and human PC-PLC.

### SUMMARY OF THE INVENTION

This invention is directed to compounds and methods for the isolation and further characterization of eukaryotic, mammalian, and/or human PC-PLC. The inventors have discovered novel D609 xanthate analogues compounds and method of synthesizing the D609 xanthate analogues compounds. In addition, the present invention discloses methods for the synthesis of an affinity chromatography resin for the isolation and purification of biologically active proteins, including eukaryotic PC-PLC.

Since most of all the commercially available affinity chromatography media requires the existence of a free amino function for coupling to the affinity column matrix, it is desirable to synthesize an amino substituted norbornyl alcohol (the alcohol to be functionalized to the xanthate or other groups as defined herein). Synthesis of an appropriately substituted D609 or D609-like molecule or D609 analogue amenable to coupling to an affinity column matrix is not a trivial task due to multiple requirements in a compound of a xanthate functional group, a bicyclic (or tricyclic) carbon core and the desirability for a primary amino group (for coupling to an affinity matrix). Amino xanthates tend to undergo self reactivity and are unstable (see U.S. Pat. No. 5,041,599).

Thus D609 analogues, such as pharmacophoric norbornanes or its analogues of the invention, are useful to prepare the complexes of this invention. These analogues include compounds of formula III and IV described herein. The amino compounds are first coupled to the appropriate resin followed by reaction of the remaining alcohol function using basic carbon disulfide to make the resin-bound xanthates.

The requirement to perform the xanthate-forming step in the presence of resin requires that the matrix be stable to organic solvent and basic conditions, which exclude polyacrylamide and agarose resins. The cross-linked agarose (Sepharose) is known to be stable at high pH and with organic solvents. Thus, the affinity ligands, such as norbornyl compounds of the invention, can be coupled to commercially available epoxy-activated Sepharose 6B (Amersham) and/or CNBr-activated Sepharose 4B (Amersham) using standard coupling techniques. The functionalized resins are then subjected to base/carbon disulfide to generate the corresponding xanthates. Alternatively, the affinity ligands containing the xanthates or other X or Y groups of the invention are coupled with the resin. The synthesis of appropriately substituted norbornanes and D-609 like molecules amenable for coupling to an affinity column matrix is shown in the examples below.

One aspect of the invention is directed to compounds of formula I and II:

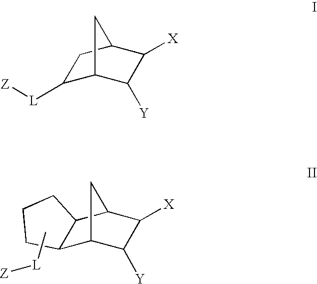

wherein;
either X or Y is selected from the group consisting of

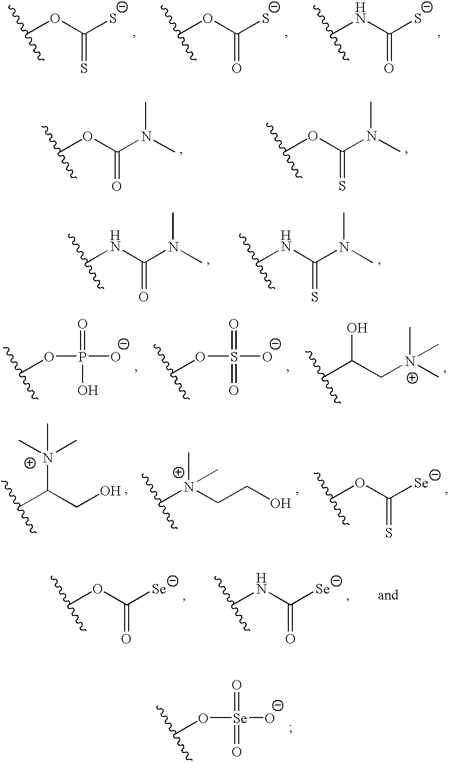

the other of X or Y is hydrogen;
L is a linker; and
Z is a solid support,
or a stereoisomer or salt thereof.

In another aspect, the invention is directed to precursor or intermediate compounds to the compounds of formula I or II. In this embodiment, the compounds lack the linker and the solid support and are of formula III or IV:

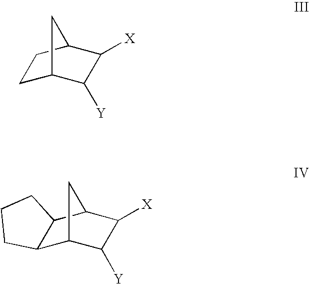

wherein X and Y are selected from the group consisting of

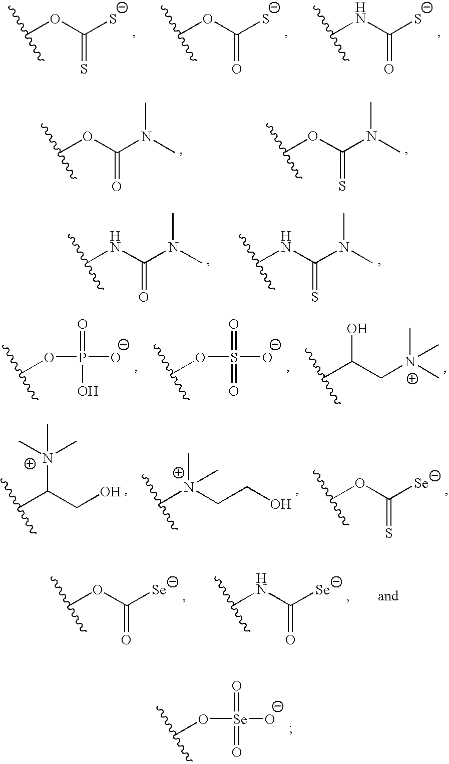

and
the other of X or Y is hydrogen,
or a stereoisomer or salt thereof,
with the proviso that when the compound is of the formula IV, X or Y is not

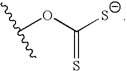

In one aspect, there is provided a compound of formula IX or XIV:

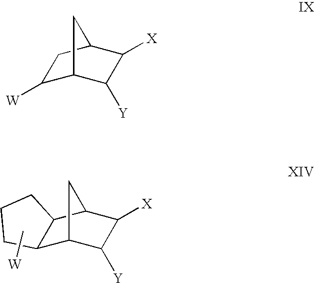

wherein;
either X or Y is selected from the group consisting of

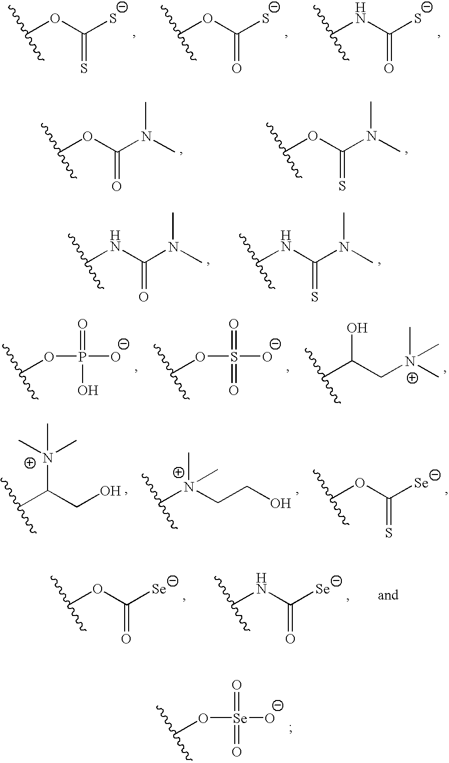

the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof.

It is contemplated that the compounds of formula I, II, III and IV are capable of complexing with eukaryotic PC-PLC.

Another aspect of the invention is directed to a method of preparing a compound of formula I:

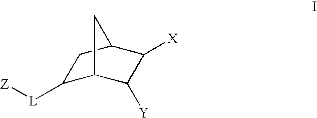

comprising contacting a compound of formula V or VI

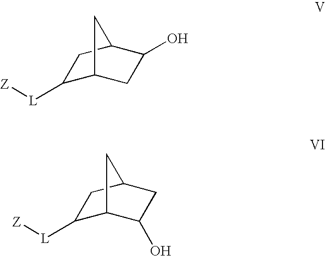

under reaction conditions to form the compound of formula I.

In some embodiments, the compound of formula I is contacted with carbon disulfide to result in the compound of formula V and VI. The reaction conditions of the above defined step include, but are not limited to, basic conditions such as sodium hydroxide or potassium hydroxide in a suitable solvent such as alcohol, acetonitrile or the like. Such solvents are well known to the skilled artisan.

The compound of formula V or VI is prepared by contacting under tethering conditions, a compound of formula VII or VIII,

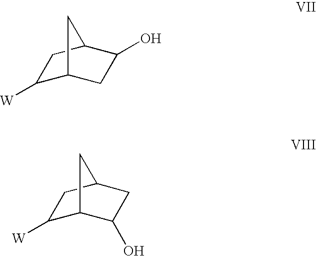

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20.

In some embodiments, the above recited tethering conditions include click chemistry defined herein.

In another embodiment of the invention, the compound of formula I may be prepared by contacting under tethering conditions, a compound of formula IX

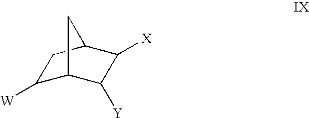

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
under reaction conditions to form the compound of formula I.

In some embodiments, the above recited tethering conditions include click chemistry defined herein.

The compound of formula IX may be prepared by comprising contacting a compound of formula VII or VIII

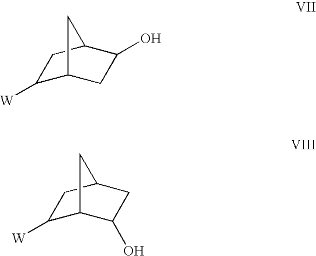

wherein W is as defined herein and the reaction is performed under conditions to form the compound of formula I.

In some embodiments, the compound of formula VII or VIII is contacted with carbon disulfide to result in the compound of formula IX. The reaction conditions of the above defined step include, but are not limited to, basic conditions such as sodium hydroxide or potassium hydroxide in a suitable solvent such as alcohol, acetonitrile or the like. Such solvents are well known to the skilled artisan.

The invention is also directed to a method of preparing a compound of formula II:

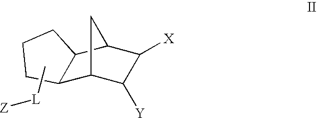

comprising contacting a compound of formula X or XI

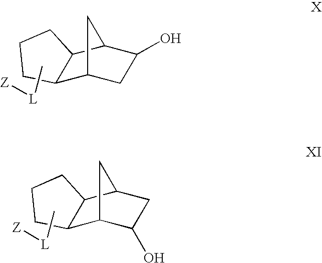

under reaction conditions to form the compound of formula II.

In some embodiments, the compound of formula X or XI is contacted with carbon disulfide to result in the compound of formula II. The reaction conditions of the above defined step include, but are not limited to, basic conditions such as sodium hydroxide or potassium hydroxide in a suitable solvent such as alcohol, acetonitrile or the like. Such solvents are well known to the skilled artisan.

The compound of formula X or XI is prepared by contacting under tethering conditions, a compound of formula XII or XIII,

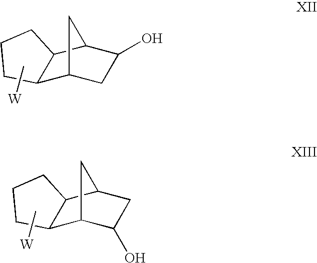

wherein W is as defined herein.

In some embodiments, the above recited tethering conditions include click chemistry defined herein.

Also provided is a method of preparing a compound of formula II:

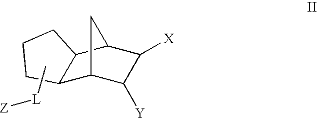

which is prepared by contacting under tethering conditions, a compound of formula XIV

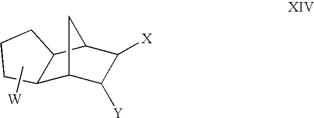

under reaction conditions to form the compound of formula I.

In some embodiments, the above recited tethering conditions include click chemistry defined herein.

The compound of formula XIV is prepared by contacting a compound of formula XII or XIII

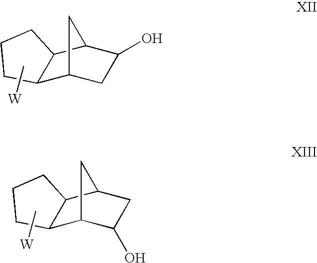

under reaction conditions to form the compound of formula I.

In some embodiments, the compound of formula XII or XIII is contacted with carbon disulfide to result in the compound of formula XIV. The reaction conditions of the above defined step include, but are not limited to, basic conditions such as sodium hydroxide or potassium hydroxide in a suitable solvent such as alcohol, acetonitrile or the like. Such solvents are well known to the skilled artisan.

In one embodiment, all of the methods of preparation described herein include the further step of isolating the product.

This invention is also directed a method of isolating eukaryotic PC-PLC. The method comprises:

a. contacting a cell homogenate comprising eukaryotic PC-PLC with a compound of any of claims 1-9 under conditions to form a complex between the compound and PC-PLC;

b. releasing mammalian PC-PLC from the complex, thereby isolating said PC-PLC.

In one embodiment, the cell homogenate is assayed to determine the presence of eukaryotic PC-PLC prior to contacting the homogenate with the compound.

In one embodiment, the conditions are affinity chromatography conditions. In this embodiment, the conditions comprise a buffered solution. In one embodiment, the complex is released by adjusting the conditions to release the PC-PLC from the complex. In some embodiments either the pH and/or the ion strength of the conditions (or solutions) are altered. The pH of the solution can be from about 6.5 to about 7.5 or 7.0. In one embodiment, the pH is adjusted by at least about 0.5 to release the PC-PLC from the complex.

In one embodiment, the method comprises recovering uncomplexed PC-PLC before releasing PC-PLC from the complex.

In one embodiment, the method comprises purifying the PC-PLC by high pressure liquid chromatography to provide substantially pure PC-PLC.

In one embodiment, the method further comprises determining the amino acid sequence of isolated PC-PLC. In another embodiment, the method further comprises determining polynucleotide sequence encoding mammalian PC-PLC. In another embodiment, the method further comprises characterizing PC-PLC by mass spectrometry.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of organic chemistry, biochemistry, microbiology, cell biology and immunology, which are within the skill of the art. Such techniques are explained fully in the literature. These methods are described in the following publications. See, e.g., the series METHODS IN ENZYMOLOGY (Academic Press, Inc.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); and ANIMAL CELL CULTURE (R. I. Freshney ed. (1987)).

As used herein, certain terms may have the following defined meanings.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “solid support” intends solid phase supports including silica gels, resins, derivatized plastic films, glass beads, glass slides, flasks, tissue culture flasks, cotton, plastic beads, alumina gels, pellets, cellulose beads, pore-glass beads, grafted co-poly beads and polyacrylamide beads. In some embodiments, the solid support is an agarose bead functionalized with free amino groups. This is referred to as “amino-functionalized agarose.” More specific examples include polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California). Solid supports also include microchips and grids. The surface of the grids may be composed of a wide variety of material including glass, plastic, silicon, gold, gelatin or nylon. Lockhart (2000) Nature, 405:827-836; Srinivas (2001) Clin. Chem., 47:1901-1911.

“D609 xanthate” or “D609” as used herein refers to compound that has the following chemical formula:

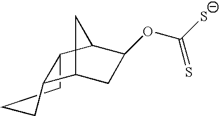

The D609 or D609 xanthate is typically a salt. Suitable salts are defined herein.

The “D609 xanthate analogue,” “xanthate analogue of D609,” or “D609 analogue,” is a compound that is an analogue of “D609” or “D609 xanthate.” For example, the analogue is a compound that differs from D609 in the number and nature of the fused rings such as, the number of rings fused together, the number of carbon atoms in the rings, or the linkage between the carbon atoms in the rings. The analogue may also differ in the nature or number of the substituents attached to the ring. For example, the D609 analogue is:

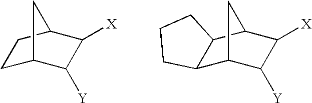

where X and Y are as defined herein.

The “norbornane” or “norbornyl” refers to bicyclo[2.2.1]heptane of the following formula:

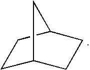

The “xanthate” refers to salts and/or esters of a xanthic acid, —OC(═S)S— or O-esters of dithiocarbonic acid where R is any organic residue.

“Linker” refers to one or more atoms comprising a chain connecting two species. More specifically, a linker refers to one or more atoms comprising a chain connecting a D-609 analogue to a solid-support by covalent bonds or by other means. A suitable “functional unit” or suitable “functional group” is a chemical moiety able to “tether” or attach the solid support to the linker via covalent or non-covalent methods. In certain embodiments, the functional unit attached to the solid support is an azide or alkynyl group which after binding with the alkynyl or the azide group of the linker, respectively, result in a linker containing a heteroaryl moiety. In some embodiments, the linkers include —(CH₂)ₘ—, —(CRR¹)ₘ—, —(CRR¹)ₘ—NR—, —(CRR¹)ₘ—O—, —(CRR¹)ₘ—O—(CRR¹)ₘ—O—, —(CRR¹)ₘ—S—, —(CRR¹)ₘ—C(O)—, —(CRR¹)ₘ—CO₂—, —(CRR¹)ₘ—S₂—, —(CRR¹)ₘ—OP(O)(OH)O—, —(CRR¹)ₘ—C(O)—NR—, —(CRR¹)ₘ—NRC(O)—, —(CRR¹)ₘ—NRC(S)—, —(CRR¹)ₘ—OC(O)—, —(CRR¹)ₘ—SO₂NR—, —(CRR¹)ₘ—NRC(O)NR—, —(CRR¹)ₘ—NRC(S)NR—, —(CRR¹)ₘ—OC(O)NR—(CRR¹)ₘ—O—(CRR¹)ₘ—O—CH(OH)—, or —(CRR¹)ₘ—O—(CRR¹)ₘ-heteroaryl; wherein R and R¹ are independently hydrogen, halo, hydroxyl, alkyl, alkoxy, or heteroaryl; and m is 0-20.

The term “alkyl” refers to a monovalent saturated aliphatic hydrocarbyl groups having from 1 to 6 carbon atoms and preferably 1 to 3 carbon atoms. This term is exemplified by groups such as methyl, ethyl, n-propyl, iso-propyl, n-butyl, t-butyl, n-pentyl and the like.

Other chemical terms used herein, such as halo, hydroxyl, and alkoxy are defined as follows.

“Halo” or “halogen” refers to fluoro, chloro, bromo and iodo and preferably is fluoro or chloro.

“Hydroxyl” refers to —OH.

“Alkoxy” refers to the term “—O-alkyl”.

“Heteroaryl” refers to a 5 or 6 membered aromatic ring containing 1-3 heteroatoms selected from N, S or O. The examples of heteroaryl include, but are not limited to, triazoles, imidazoles, pyrroles, oxadiazoles, thiadiazoles, and the like.

“Stereoisomer” or “stereoisomers” refer to compounds that differ in the chirality of one or more stereocenters. Stereoisomers include enantiomers and diastereomers.

“Salt” refers to the salt of a compound, which is derived from a variety of organic and inorganic counter ions well known in the art and include, by way of example only, sodium, potassium, calcium, magnesium, ammonium, and tetraalkylammonium, and other positively charged ions.

“Affinity chromatography” is a chromatographic method of separating biochemical mixtures, based on a highly specific biologic interaction such as that between antigen and antibody, enzyme and substrate, or receptor and ligand.

A “control” is an alternative subject or sample used in an experiment for comparison purpose. A control can be “positive” or “negative”. For example, where the purpose of the experiment is to determine a correlation of the binding of a ligand to a receptor, it is generally preferable to use a positive control (a subject or a sample from a subject, for which the ligand has high affinity and avidity for the binding partner), and a negative control (a subject or a sample from a subject for which the affinity and avidity is low or non-existent).

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

“Eurkaryotic” cells comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, y east, higher plant, insect and mammalian cells, or alternatively from a prokaryotic cells as described above. Non-limiting examples include simian, bovine, porcine, murine, rats, avian, reptilian and human.

A “composition” is intended to mean a combination of active agent and another compound or composition, inert (for example, a detectable agent or label) or active, such as an adjuvant.

The “ionic strength” of a solution is a function of the concentration of all ions present in the solution. “Buffered solutions” are solutions of a desired pH can be prepared with various ionic strengths using, for example, phosphate buffer saline (abbreviated PBS). PBS is prepared from, sodium phosphate and potassium phosphate. It is contemplated that a suitable range of phosphate ion concentration is from about 25 to 100 mM for the present invention.

The term “substantially pure” is intended to mean PC-PLC which has been separated from at least some of those components which naturally accompany it. Typically, a protein is substantially pure when it is at least 60% (by weight) free from the proteins and other naturally-occurring organic molecules with which it is naturally associated in vivo. Preferably, the purity (by weight) is at least 75%, more preferably at least 90%. and most preferably at least 99%. Purity can be measured by any appropriate method, e.g., column chromatography, polyacrylamide gel electrophoresis, or high-performance liquid chromatography (HPLC) analysis.

The term “isolate” means separated from constituents, such as starting reagents, in which the compound or reaction product might be in contact with.

The present invention discloses methods for the synthesis of an affinity chromatography resin for the isolation and purification of biologically active proteins.

### Compositions and Methods of Making

This invention provides a complex that is useful to purify eukaryotic, more specifically mammalian, and still more specifically human PC-PLC. The complex comprises a solid support covalently attached to a xanthate analogue of D609 optionally via a linker to the solid support such as a chromatograpy bead. In some embodiments, the linker is, but not limited to, a saturated alkyl chain, a polyethylene glycol, or a polyfluorinated alkyl chain. In some embodiments, the purpose of the linker is to 1) tether the recognized molecular moiety to the solid support and 2) to extend the recognized molecular moiety away from the bulk surface of the solid support. These functions of the linker serve to allow specific binding to the molecular moiety that is recognized by PC-PLC. Unlike prior art compositions and methods, the invention described herein is the synthesis on the bead of the xanthate analogue. This new technology in turn allows a simpler and efficient isolation and purification of eukaryotic, and specifically mammalian, PC-PLC attached to the bead and the development of specific novel inhibitors for PC-PLC.

The invention is directed to compounds that are D609-like compounds attached to a solid support, optionally through a linker.

The compounds contemplated by this invention are of formula I and II.

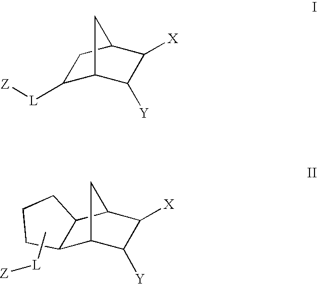

wherein;
either X or Y is selected from the group consisting of

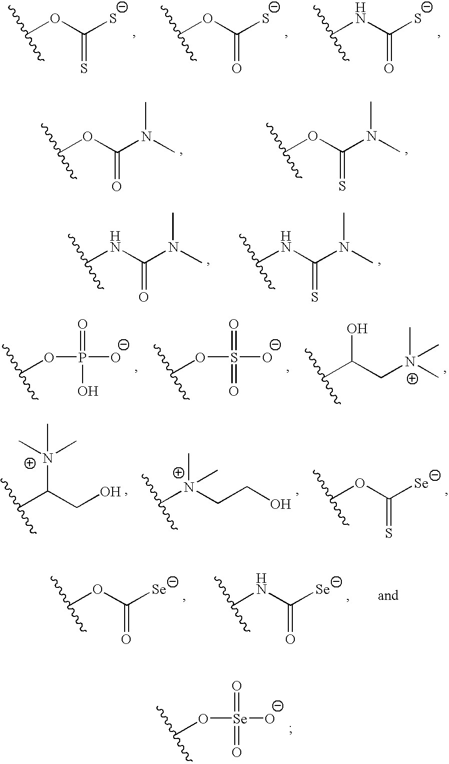

the other of X or Y is hydrogen;
L is a linker; and
Z is a solid support,
or a stereoisomer or salt thereof.

Stereoisomers contemplated by this invention include stereoisomers of the X, Y, and L groups. Specific examples of the X and Y stereoisomers are shown below.

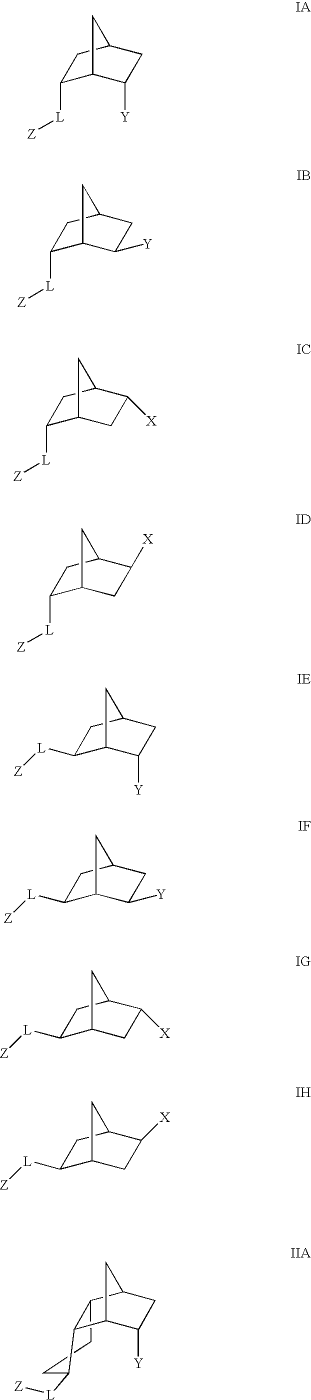

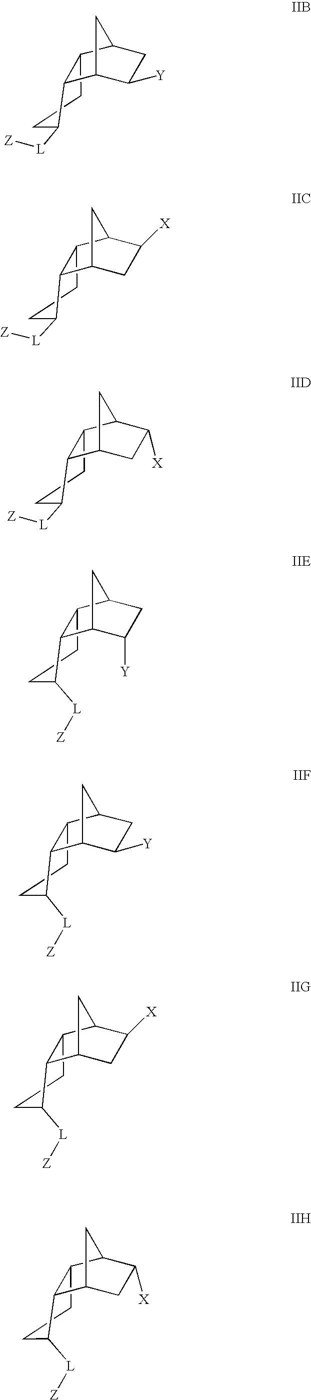

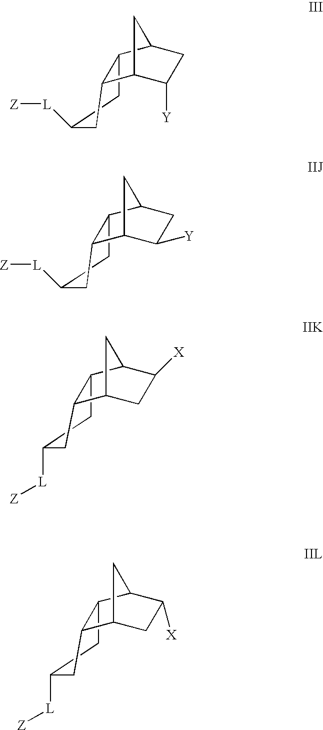

It is further contemplated that the compounds of formula I and II may be further derivatized to incorporate a choline group at the X or Y position. This can be achieved by substituting one or more atoms of the X or Y group, such as a hydrogen, with choline. Choline has the following chemical structure:

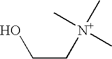

The choline group may be bound to the X or Y through replacement of one of the hydrogen atoms on the hydroxy group, one of the methyl groups on the amino, or the ethylene chain of the choline.

In some embodiments of the invention, X or Y is selected from the group consisting of

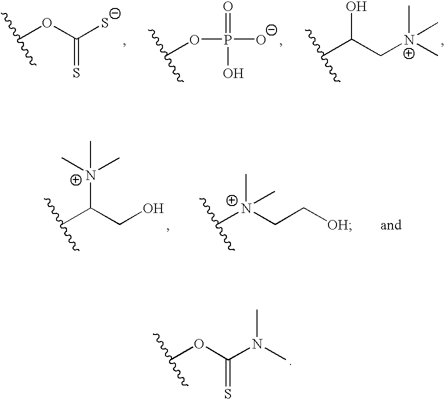

In some embodiments of the invention, X or Y is selected from the group consisting of

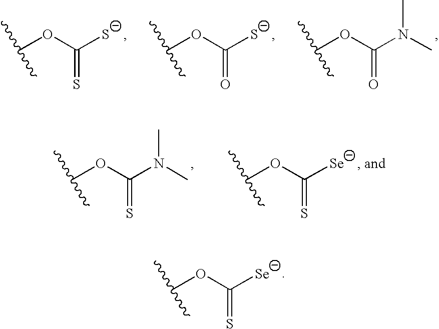

In some embodiments of the invention, X or Y is selected from the group consisting of

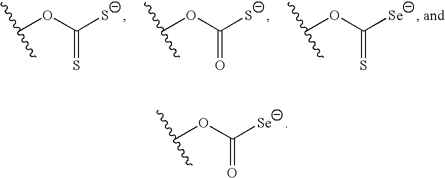

In some embodiments of the invention, X or Y is selected from the group consisting of

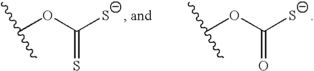

In other embodiments, X or Y is

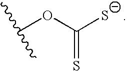

In some embodiments of the invention, X or Y is selected from the group consisting of

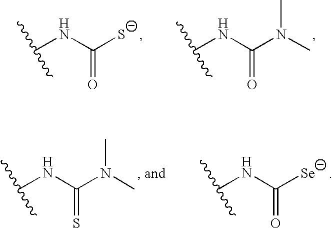

In some embodiments of the invention, X or Y is selected from the group consisting of

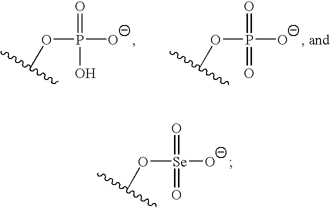

In some embodiments of the invention, X or Y is selected from the group consisting of

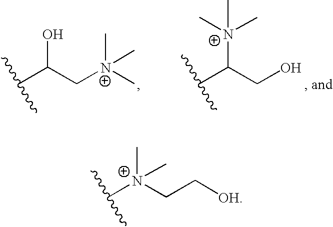

In one embodiment, Z is selected from the group consisting of silica gels, resins, derivatized plastic films, glass beads, glass slides, flasks, tissue culture flasks, cotton, plastic beads, alumina gels, pellets, cellulose beads, pore-glass beads, grafted co-poly beads and polyacrylamide beads.

In one embodiment, Z is selected from the group consisting of polystyrene, polystyrene resin grafted with polyethylene glycol, polyamide resin, polyacrylamide resin, polydimethylacrylamide resin, silica, dextran, agarose, and a cross-linked agarose. In another embodiment, Z is agarose. In another embodiment, Z is cross-linked agarose. In another embodiment, Z is amino-functionalized agarose.

In one embodiment, L is a suitable functional unit to tether Z. In one embodiment, L is selected from the group consisting of —(CH₂)ₘ—, —(CRR¹)ₘ—NR—, —(CRR¹)ₘ—O—, —(CRR¹)ₘ—O—(CRR¹)ₘ—O—, —(CRR¹)ₘ—S—, —(CRR¹)ₘ—C(O)—, —(CRR¹)ₘ—S₂—, —(CRR¹)ₘ—OP(O)(OH)O—, —(CRR¹)ₘ—C(O)—NR—, —(CRR¹)ₘ—SO₂NR—, —(CRR¹)ₘ—NRC(O)NR—, —(CRR¹)ₘ—NRC(S)NR—, and —(CRR¹)ₘ—OC(O)NR; where each of R and R¹ are independently selected from the group consisting of H, alkyl, and heteroaryl; and m is 0-20.

In one embodiment, L has a suitable functional unit to tether Z. In one embodiment, L is selected from the group consisting of —(CH₂)ₘ—, —(CRR¹)ₘ—, —(CRR¹)ₘ—, —NR—, —(CRR¹)ₘ—O—, —(CRR¹)ₘ—O—(CRR¹)ₘ—O—, —(CRR¹)ₘ—S—, —(CRR¹)ₘ—C(O)—, —(CRR¹)ₘ—, —CO₂—, —(CRR¹)ₘ—S₂—, —(CRR¹)ₘ—OP(O)(OH)O—, —(CRR¹)ₘ—C(O)—NR—, —(CRR¹)ₘ—NRC(O)—, —(CRR¹)ₘ—NRC(S)—, —(CRR¹)ₘ—OC(O)—, —(CRR¹)ₘ—SO₂NR—, —(CRR¹)ₘ—NRC(O)NR—, —(CRR¹)ₘ—NRC(S)NR—, —(CRR¹)ₘ—OC(O)NR—, —(CRR¹)ₘ—O—(CRR¹)ₘ-heteroaryl, and —(CRR¹)ₘ—O—(CRR¹)ₘ—O—CH(OH)—; where each of R and R¹ are independently selected from the group consisting of hydrogen, halo, hydroxyl, alkyl, alkoxy, and heteroaryl; and m is 0-20.

In one embodiment, L is —(CRR¹)ₘ— where R and R¹ are independently selected from the group consisting of hydrogen, halo, hydroxyl, alkyl, alkoxy, and heteroaryl; and m is 0-20.

In one embodiment, L is —(CRR¹)ₘ— where R and R¹ are independently selected from the group consisting of hydrogen, halo, and hydroxyl; and m is 0-10.

In one embodiment, L is —(CRR¹)ₘ— where R and R¹ are independently selected from the group consisting of hydrogen, halo, and hydroxyl; and m is 0-5.

In one embodiment, L is —(CRR¹)ₘ— where R and R¹ are independently selected from the group consisting of hydrogen or halo; and m is 0-5, 0-4 or 0-3 or 0-2 or 0-1 or 1 or 2 or 3 or 4 or 5.

In one embodiment, L is —(CRR¹)ₘ— where R and R¹ are independently selected from the group consisting of hydrogen or hydroxyl; and m is 0-5, 0-4 or 0-3 or 0-2 or 0-1 or 1 or 2 or 3 or 4 or 5.

In one embodiment, L is —(CRR¹)ₘ— where R and R¹ are independently selected from the group consisting of hydrogen or hydroxyl; and m is 3, 2, or 1.

In one embodiment, L is —(CRR¹)ₘ—O— where R and R¹ are independently selected from the group consisting of hydrogen, halo, hydroxyl, alkyl, alkoxy, and heteroaryl; and m is 0-20.

In one embodiment, L is —(CRR¹)ₘ—O— where R and R¹ are independently selected from the group consisting of hydrogen, halo, and hydroxyl; and m is 0-10.

In one embodiment, L is —(CRR¹)ₘ—O— where R and R¹ are independently selected from the group consisting of hydrogen, halo, and hydroxyl; and m is 0-5.

In one embodiment, L is —(CRR¹)ₘ—O— where R and R¹ are independently selected from the group consisting of hydrogen or halo; and m is 0-5, 0-4 or 0-3 or 0-2 oro-1 or 1 or 2 or 3 or 4 or 5.

In one embodiment, L is —(CRR¹)ₘ—O— where R and R¹ are independently selected from the group consisting of hydrogen or hydroxyl; and m is 0-5, 0-4 or 0-3 or 0-2 or 0-1 or 1 or 2 or 3 or 4 or 5.

In one embodiment, L is —(CRR¹)ₘ—O— where R and R¹ are independently selected from the group consisting of hydrogen or hydroxyl; and m is 3, 2, or 1.

In one embodiment, L is —(CRR¹)ₘ—O—(CRR¹)ₘ-heteroaryl, where R and R¹ are independently selected from the group consisting of hydrogen, halo, hydroxyl, alkyl, alkoxy, and heteroaryl; and m is independently 0-20.

In one embodiment, L is —(CRR¹)ₘ—O—(CRR¹)ₘ-heteroaryl, where R and R¹ are independently selected from the group consisting of hydrogen, halo, and hydroxyl; and m is independently 0-10.

In one embodiment, L is —(CRR¹)ₘ—O—(CRR¹)ₘ-heteroaryl where R and R¹ are independently selected from the group consisting of hydrogen, halo, and hydroxyl; and m is independently 0-5.

In one embodiment, L is —(CRR¹)ₘ—O—(CRR¹)ₘ-heteroaryl where R and R¹ are independently selected from the group consisting of hydrogen or halo; and m is independently 0-10, or 5-15, or 0-5, or 0-4 or 0-3 or 0-2 or 0-1 or 1 or 2 or 3 or 4 or 5.

In one embodiment, L is —(CRR¹)ₘ—O—(CRR¹)ₘ-heteroaryl where R and R¹ are independently selected from the group consisting of hydrogen or hydroxyl; and m is independently 5-15, or 0-10, or 0-5, or 0-4 or 0-3 or 0-2 or 0-1 or 1 or 2 or 3 or 4 or 5.

In one embodiment, L is —(CRR¹)ₘ—O—(CRR¹)ₘ-heteroaryl where R and R¹ are independently selected from the group consisting of hydrogen or hydroxyl; and m is independently 10, 8, 9, 7, 6, 5, 4, 3, 2, or 1.

In one embodiment with respect to each of the above noted embodiments, m is 0-4 or alternatively 0-3 or alternatively 0-2 or alternatively, 0-1 or alternatively 1 or 2. In one aspect, m is 5-20, or alternatively 5-10, or alternatively 0-10, or alternatively 10-20, or alternatively 8-20, or alternatively 5-15, or alternatively 12-20, or alternatively 15-20 or alternatively 2-10 or alternatively 2-15.

In one embodiment, R and R¹ are independently selected from the group consisting of hydrogen, halo, and hydroxyl. In one embodiment, R and R¹ are independently selected from the group consisting of hydrogen, alkyl, alkoxy, and heteroaryl. In one embodiment, R and R¹ are independently selected from hydrogen or alkyl. In one embodiment, R and R¹ are independently selected from hydrogen or halo.

In one embodiment, R and R¹ are independently selected from hydrogen, halo or hydroxyl. In one embodiment, R and R¹ are independently selected from hydrogen or hydroxyl.

In one embodiment, L is —(CH₂)ₘ— or —(CH₂)ₘ—O—; and m is 8-20.

In one embodiment, the compound is of formula I:

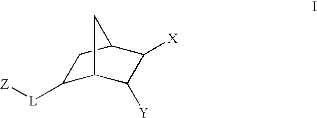

wherein;

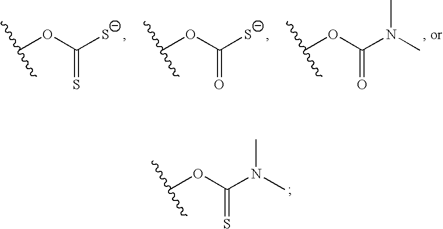

X is
Y is hydrogen;
L is —(CH₂)ₙ— or —(CH₂)ₙ—O—;
n is 8-20;
and Z is agarose;
or a stereoisomer or salt thereof.

In one embodiment, the compound is of formula I:

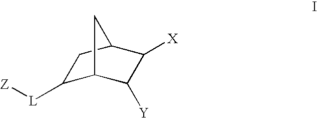

wherein;
X is

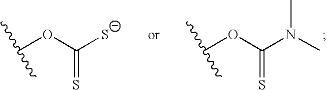

Y is hydrogen;
L is —(CH₂)ₙ— or —(CH₂)ₙ—O—;
n is 8-20;
and Z is agarose;
or a stereoisomer or salt thereof.

In one embodiment, the compound is of formula I:

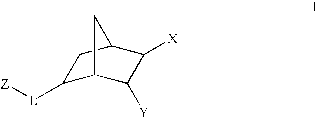

wherein;
X is

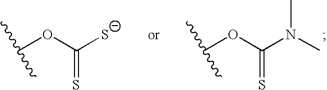

Y is hydrogen;
L is —(CH₂)ₙ— or —(CH₂)ₙ—O—;
n is 5-10;
and Z is agarose;
or a stereoisomer or salt thereof.

In one embodiment, the compound is of formula I:

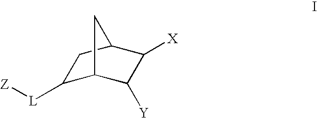

wherein;
Y is

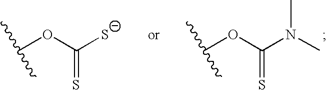

X is hydrogen;
L is —(CH₂)ₙ— or —(CH₂)ₙ—O—;
n is 8-20;
and Z is agarose;
or a stereoisomer or salt thereof.

It is contemplated that these compounds will bind to eukaryotic PC-PLC and thus be useful in isolating, purifying, and/or identifying eukaryotic PC-PLC.

Also contemplated by this invention are precursor or intermediate compounds to compounds of formula I and II. These compounds include compounds of formula III and IV.

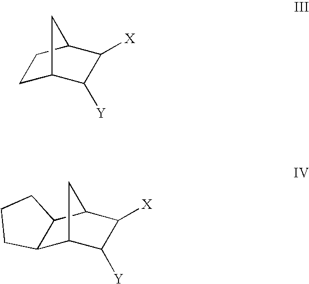

wherein X and Y are selected from the group consisting of

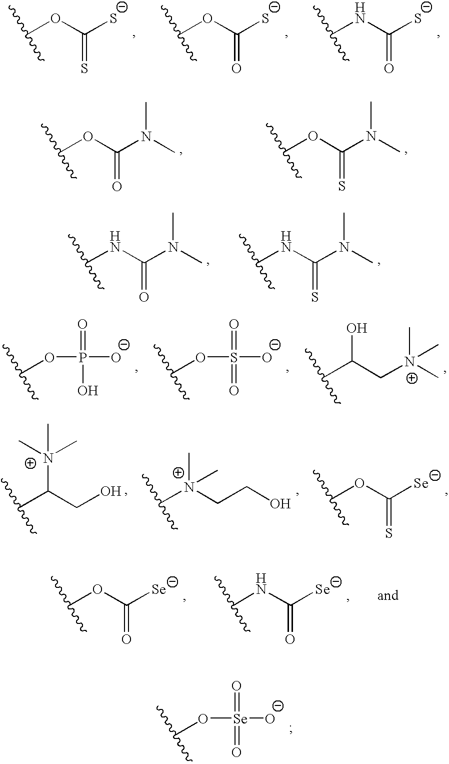

and
the other of X or Y is hydrogen,
or a stereoisomer or salt thereof,
with the proviso that when the compound is of the formula IV, X or Y is not

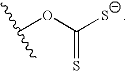

In one aspect, there is provided a compound of formula IX or XIV:

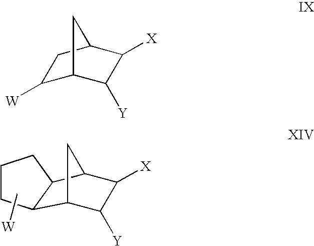

wherein;
either X or Y is selected from the group consisting of

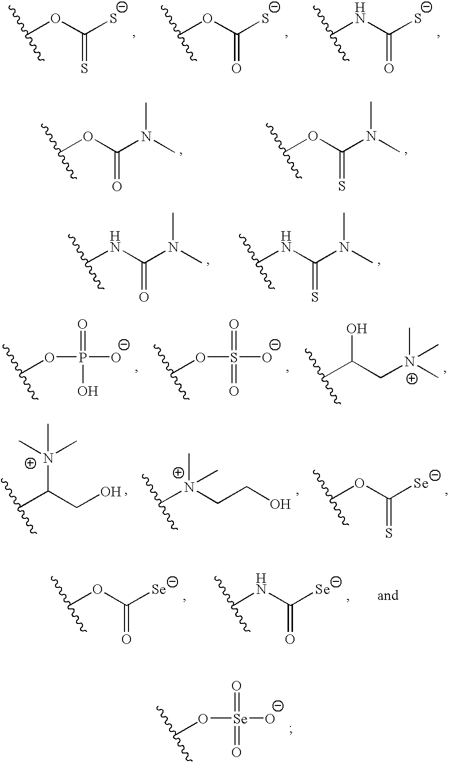

the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof.

In one embodiment of the compound of formula III, IV, IX, or XIV, the X or Y is selected from the group consisting of:

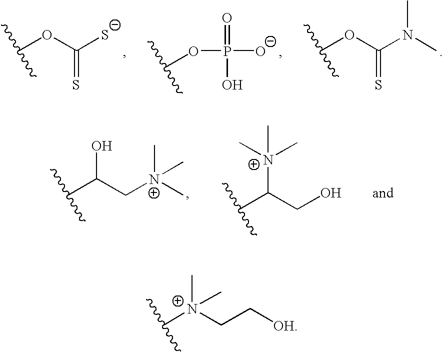

In one embodiments of the compound of formula III, IV, IX, or XIV, X or Y is selected from the group consisting of

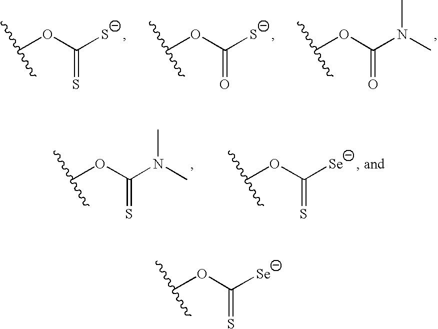

In one embodiment of the compound of formula III, IV, IX, or XIV, X or Y is selected from the group consisting of

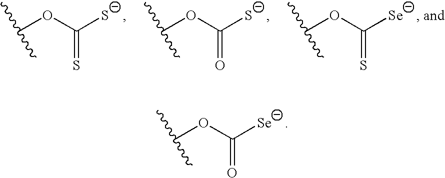

In one embodiment of the compound of formula III, IV, IX, or XIV, X or Y is selected from the group consisting of

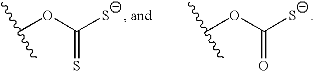

In other embodiments of the compound of formula III, IV, IX, or XIV, X or Y is

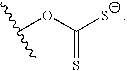

In one embodiment of the compound of formula III, IV, IX, or XIV, X or Y is selected from the group consisting of

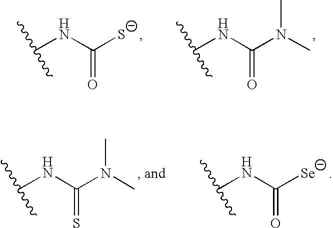

In one embodiment of the compound of formula III, IV, IX, or XIV, X or Y is selected from the group consisting of

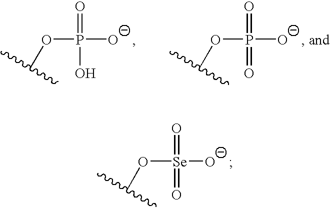

In one embodiment of the compound of formula III, IV, IX, or XIV, X or Y is selected from the group consisting of

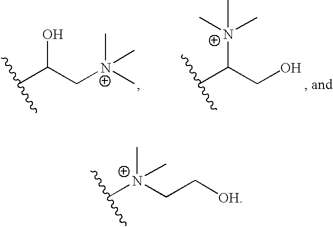

In another embodiment of the compound of formula III, IV, IX, or XIV, X or Y is

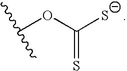

In one embodiment of the compounds of formula III, IV, IX, or XIV, one of X or Y is a xanthate and other of X and Y is a hydrogen; and W is —(CRR¹)ₘ—NH₂, —(CRR¹)ₘ—N₃, —(CRR¹)ₘ—O—(CRR¹)ₘ—N₃, —(CRR¹)ₘ—C≡CH, —(CRR¹)ₘ—O—(CRR¹)ₘ—C≡CH, or —(CRR¹)ₘ—O—(CRR¹)ₘ—OH where m is 0-10 and R and R¹ are independently selected from hydrogen or alkyl.

In one embodiment of the compounds of formula III, IV, IX, or XIV, one of X or Y is a xanthate, carbamate, or thiocarbamate, and other of X and Y is a hydrogen; and W is —(CRR¹)ₘ—NH₂, —(CRR¹)ₘ—N₃, —(CRR¹)ₘ—O—(CRR¹)ₘ—N₃, —(CRR¹)ₘ—C≡CH, —(CRR¹)ₘ—O—(CRR¹)ₘ—C≡CH, or —(CRR¹)ₘ—O—(CRR¹)ₘ—OH where m is 0-10 and R and R¹ are independently selected from hydrogen or alkyl.

In one embodiment of the compounds of formula I or II, L is —(CRR¹)ₘ—, —(CRR¹)ₘ—NR—, —(CRR¹)ₘ—O—(CRR¹)ₘ-heteroaryl, —(CRR¹)ₘ—O—(CRR¹)ₘ—O—CH(OH)—where m is 0-10 and R and R¹ are independently selected from hydrogen or alkyl; Z is agarose. or a cross linked agarose; and one of X or Y is a xanthate and other of X and Y is a hydrogen.

In one embodiment, W is —(CRR¹)ₘ—O—(CRR¹)ₘ—N₃ or —(CRR¹)ₘ—O—(CRR¹)ₘ—C≡CH where m is independently, 0, 1, 3, 5, 8, or 10. In one embodiment, W is —CH₂—O—(CRR¹)ₘ—N₃ or —CH₂—O—(CRR¹)ₘ—C≡CH where m is 0, 1, 3, 5, 8, or 10.

The invention is also directed to methods of making the compounds of formula I and II.

In one aspect, there is provided a method of preparing a compound of formula I:

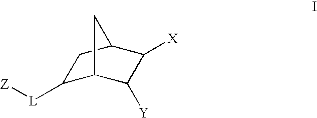

wherein;
either X or Y is selected from the group consisting of

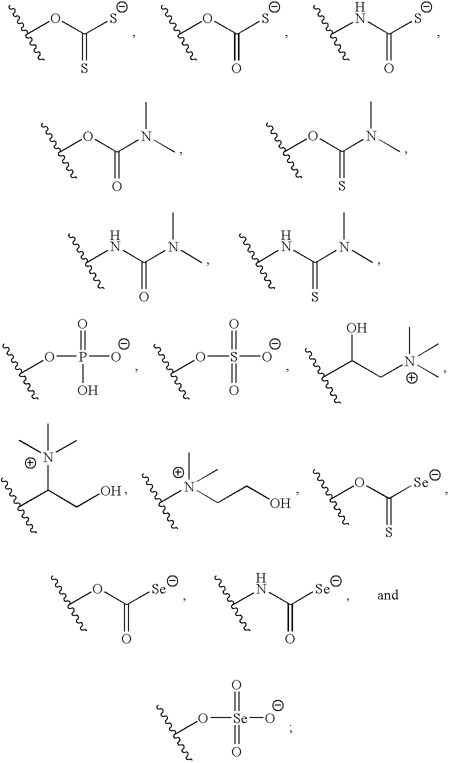

the other of X or Y is hydrogen;
L is a linker; and
Z is a solid support,
or a stereoisomer or salt thereof,
comprising contacting a compound of formula V or VI

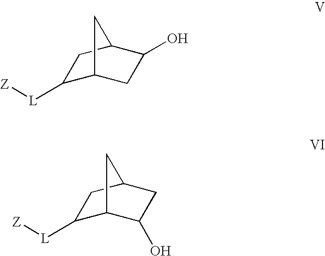

under reaction conditions to form the compound of formula I.

In some embodiments of the method provided herein, the method further comprises isolating the compound of formula I.

In some embodiments, the compound of formula V or VI:

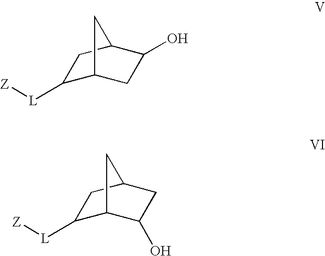

wherein L is a linker;
Z is a solid support,
or a stereoisomer or salt thereof,
is prepared by contacting under tethering conditions, a compound of formula VII or VIII,

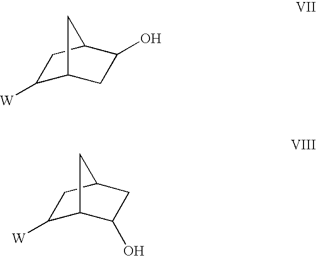

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20.

In one aspect, there is provided a method of preparing a compound of formula I:

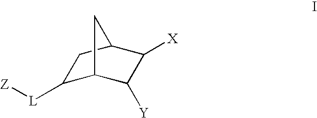

wherein either X or Y is selected from the group consisting of

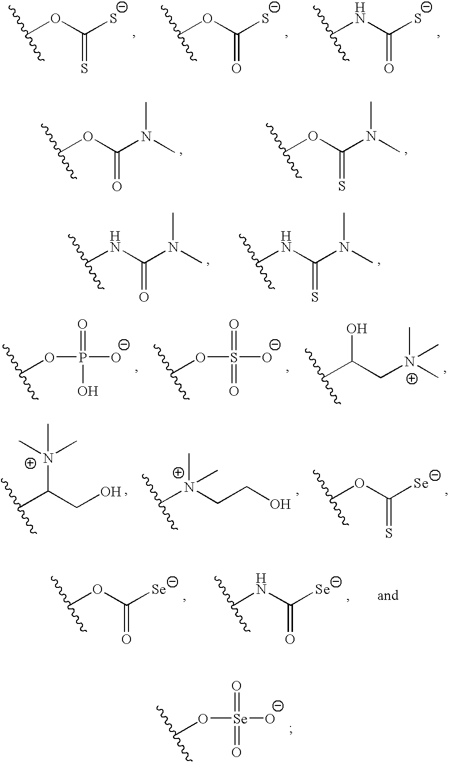

the other of X or Y is hydrogen;
L is a linker;
Z is a solid support,
or a stereoisomer or salt thereof,
by contacting under tethering conditions, a compound of formula IX

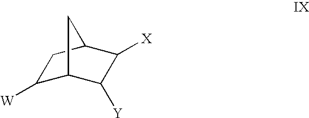

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)_{m OC(O)X, —(CRR}¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
under reaction conditions to form the compound of formula I.

In one aspect, there is provided a method of preparing a compound of formula IX:

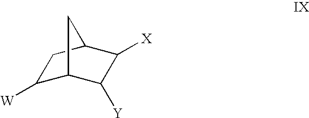

wherein;
either X or Y is selected from the group consisting of

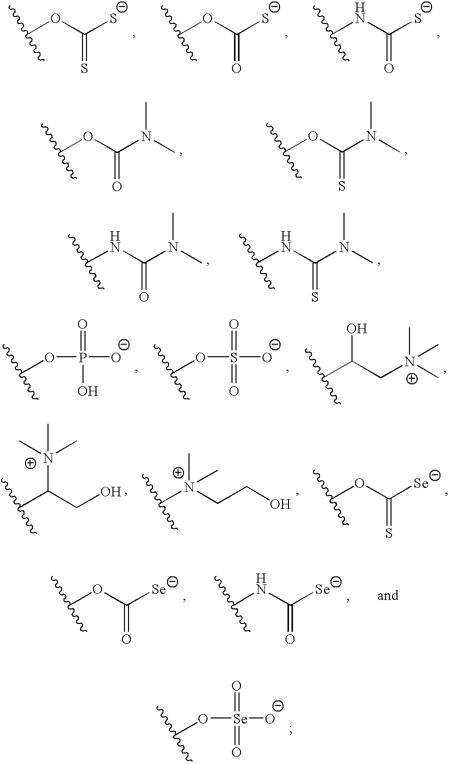

the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof,
comprising contacting a compound of formula VII or VIII

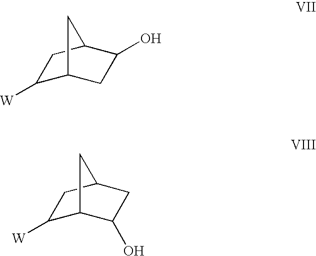

under reaction conditions to form the compound of formula I.

In one aspect, there is provided a method of preparing a compound of formula II:

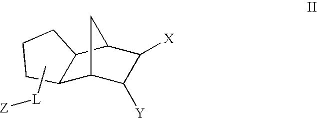

wherein;
either X or Y is selected from the group consisting of

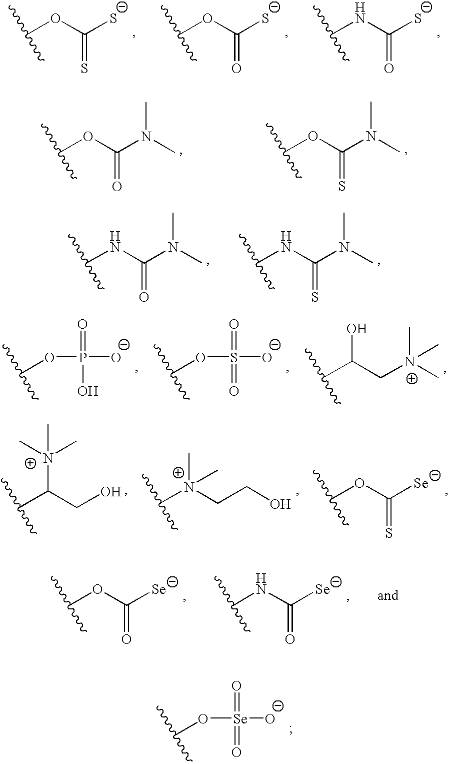

the other of X or Y is hydrogen;
L is a linker; and
Z is a solid support,
or a stereoisomer or a salt thereof,
comprising contacting a compound of formula X or XI

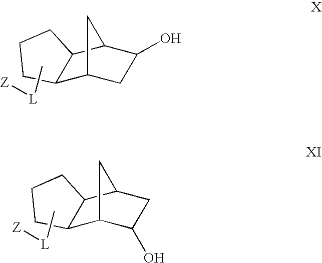

under reaction conditions to form the compound of formula II.

In some embodiments, the compound of formula X or XI:

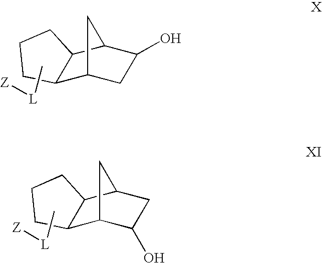

is prepared by contacting under tethering conditions, a compound of formula XII or XIII,

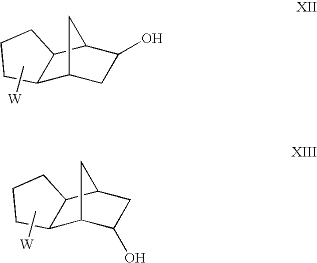

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20.

In one aspect, there is provided a method of preparing a compound of formula II:

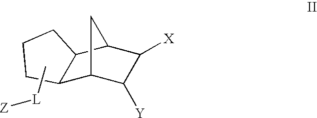

wherein either X or Y is selected from the group consisting of

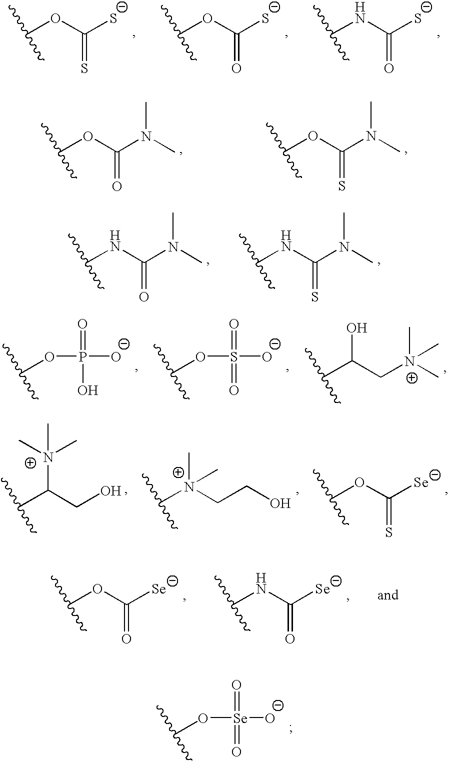

the other of X or Y is hydrogen;
L is a linker;
Z is a solid support,
or a stereoisomer or salt thereof,
is prepared by contacting under tethering conditions, a compound of formula XIV

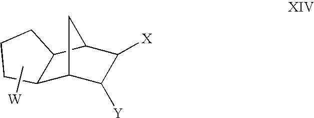

wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘOC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
under reaction conditions to form the compound of formula I.

In one aspect, there is provided a method of preparing a compound of formula XIV:

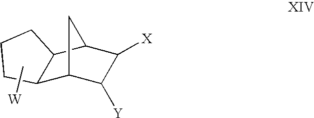

wherein;
either X or Y is selected from the group consisting of

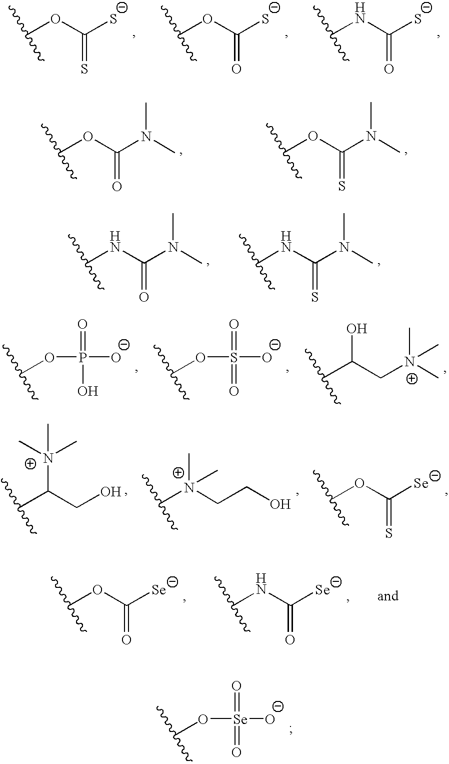

the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof,
comprising contacting a compound of formula XII or XIII

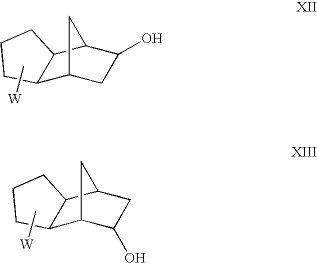

under reaction conditions to form the compound of formula I.

The compounds of this invention can be prepared from readily available starting materials using the following general methods and procedures. Unless otherwise indicated, the starting materials are commercially available and well known in the art. It will be appreciated that where typical or preferred process conditions (i.e., reaction temperatures, times, mole ratios of reactants, solvents, pressures) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

Compounds in the present invention may be better understood by the following synthetic schemes that illustrate methods for the synthesis of compounds of the invention. Unless otherwise indicated, the reagents used in the following examples are commercially available and may be purchased from vendors such as Sigma-Aldrich Company, Inc. (Milwaukee, Wis., USA) or are known in the art.

The requirement to perform the xanthate-forming step in the presence of resin requires that the matrix be stable to organic solvent and basic conditions, which exclude polyacrylamide and agarose resins. However, cross-linked agarose (Sepharose) is known to be stable at high pH and with organic solvents. Thus, the affinity ligands can be coupled to commercially available epoxy-activated Sepharose 6B (Amersham) and/or CNBr-activated Sepharose 4B (Amersham) using standard coupling techniques The functionalized resins are then subjected to base/carbon disulfide to generate the corresponding xanthates using methods known in the art, e.g., M. B. Smith and J. March, March's Advanced Organic Chemistry, Fifth Edition, Wiley, New York, 2001.

One aspect of the present invention is the tethering of a D-609 or a D-609 like molecule to a suitable solid support or resin. One possible method for the tethering is click chemistry. The term “click chemistry” is intended to mean a chemical reaction which forms product quickly and reliably by joining small units together. Examples of “click chemistry” methods include, but are not limited to, dipolar cycloaddition reactions, cycloaddition reactions and nucleophilic substitution reactions. Therefore, with the appropriately functionalized bead or the solid support and D-609 or a D-609 like molecule, one could tether the two together under various reaction conditions.

### Synthesis of an Appropriately Substituted D609 or D609-Like Molecules

The compounds of the invention may be synthesized in a manner as described in the summary of the invention. In one embodiment, the norbornane ring is built or synthesized on the bead or solid support. Once the norborane ring is assembled, the remaining functional group, such as alcohol, may be further functionalized to the xanthate-like group. In another embodiment, the functionalized norbornane ring is applied to the bead via “click” chemistry. Both methods are described below.

### Example 1

### Synthesis of an Appropriately Substituted Norbornane Molecule Amenable for Coupling to an Affinity Column Matrix

The synthesis of an appropriately substituted norbornane molecule amenable for coupling to an affinity column matrix can be prepared as follows. Bicyclo[2.2.1]hepta-2,5-diene can be reacted with potassium isothiocyanate and concentrated H₂SO₄ to form the norbornyl isothiocyanate. The free amine, exo-5-aminonorborn-2-ene, can be liberated by the addition of solid NaOH at elevated temperatures (Scheme 1a). The endo-5-aminonorborn-2-ene can synthesized from the corresponding norbornene-2-carboxylic acid. A mixture of endo- and exo-norbornene-2-carboxylic acid can be reacted with thionyl chloride (SOCl₂) followed by sodium azide (NaN₃) to yield the corresponding isocyanate. The addition of trifluoroacetic acid (TFA) forms isomeric trifluoroacetamides which can be separated to give pure endo-product. Hydrolysis with potassium carbonate (K₂CO₃) would yield the endo-5-aminonorborn-2-ene (Scammells, et al. (1996) Tetrahedron 52:13 4735-4744) (Scheme 1b). Using the general method of Hutchinson et al. (Hutchinson, et al. (1999) Bioorg. Med. Chem. Lett. 9:7 933-936) both of the 5-aminonorborn-2-enes can be protected using tert-butyl carbonate (BOC) followed by alcohol formation via hydroboration (borane/THF) and oxidation (basic H₂O₂) of the alkene. Deprotection of the BOO group using HCl and dioxane would yield a mixture of the 5- and 6-hydroxynorborn-2-yl-amine (Scheme 1c).

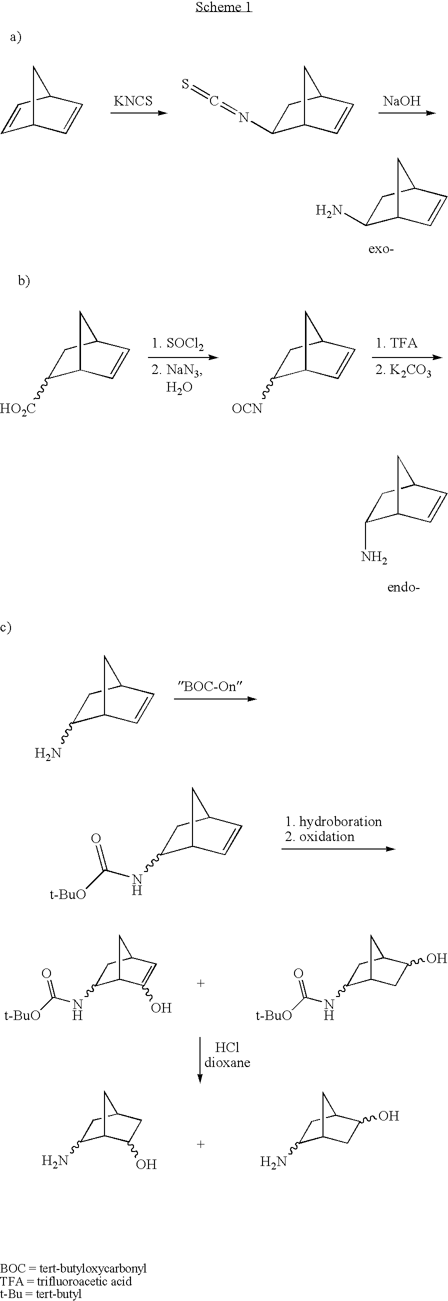

### Example 2

### Synthesis of an Appropriately Substituted D609 or D609-Like Molecule Amenable for Coupling to an Affinity Column Matrix

The synthesis of an appropriately substituted D609 or D609-like molecule amenable for coupling to an affinity column matrix can be prepared as follows. The dicyclopentadiene compounds have been synthesized using the methods discussed herein (Takano, et al. (1994) Synthesis 687; Tanaka, et al. (1995) Synthesis 1237-1239). Racemic dicyclopentadiene can be oxidized to the corresponding endo-acetates using manganese acetate with a catalytic amount of potassium bromide. The enantiomeric compounds can be resolved via kinetic resolution using lipase PS-on-Celite (Amano: Psuedomonas sp.). The resulting enantiomerically pure compounds can then be separated via column chromatography (Scheme 2a). After separation, the enantiomerically pure endo-alcohol can be oxidized with retention to the corresponding enantiomerically pure dicyclopentadiene using manganese dioxide. The endo-acetate can be hydrolyzed with potassium carbonate to liberate the alcohol, and subsequently converted to the enantiomerically pure dicyclopentadiene using manganese dioxide.

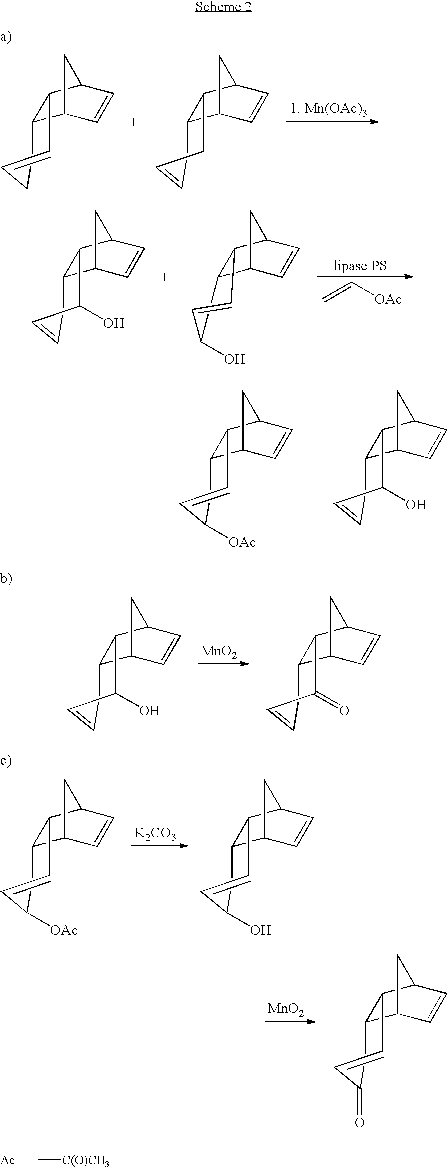

Although only one of the above enantiomeric dicyclopentadiene precursors is exemplified below, both can be taken on under identical reaction conditions to form various epimers of the amino alcohols shown in Scheme 3.

Using the methods of Nakada et al. (Nakada et al. (1997) Tetrahedron Lett. 38:5 857-860), the enantiomeric ketodicyclopentadiene precursors can be taken on to make the appropriately substituted D609 or D609-like molecule amenable for coupling to an affinity column matrix. Selective reduction of the double bond alpha to the ketone using zinc and acetic acid would give the saturated ketone that can then be transformed into the corresponding oxime by treatment with hydroxylamine in pyridine. Reduction of the oxime using lithium aluminum hydride at room temperature would afford the endo amine product. Generation of the exo-amine product can be accomplished from the ketone via a sodium borohydride reduction to the endo-alcohol, followed by azide displacement using diphenylphosphoryl azide (DPPA), (NCO₂Et₂)₂ and triphenylphosphine (PPh₃) in THF. Reduction of the azide to give the endo-amine can then be accomplished using lithium aluminum hydride at room temperature. As described in Scheme 1b, protection of the amine function by conversion to the BOO followed by hydroboration/oxidation and BOO deprotection would yield the corresponding isomeric amino alcohols that can be separated and isolated via column chromatography.

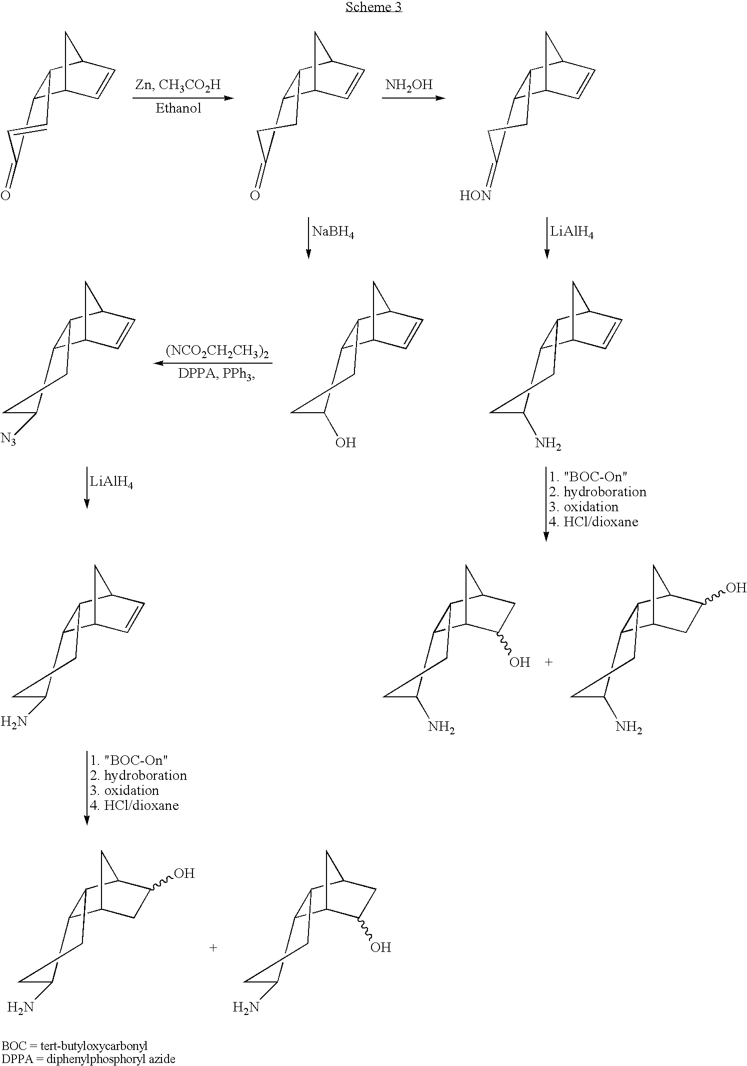

### Example 3

### Synthesis of the Xanthate Moiety

The xanthate moiety can be appended to the desired norbornane derivative or D-609 like derivative using carbon disulfide in the presence of base (M. B. Smith and J. March, March's Advanced Organic Chemistry, Fifth Edition, Wiley, New York, 2001) (Scheme 4).

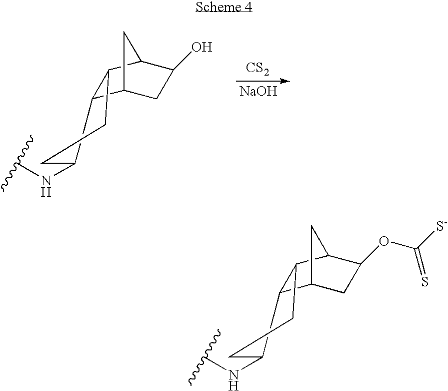

### Example 4

### Coupling Strategies Capable of Incorporating Xanthate, Thiocarbamate, and Carbamate-Functionalized Ligands

The affinity ligands can be coupled via a functionalized linkage to the solid support, by any one of a number of ‘click chemistry’ methods, some examples of which are shown in Scheme 5, below. The first two methods (Scheme 5, a and b) utilize a dipolar cycloaddition reaction (Rostovtsev, V. V., et al. (2002) Angew. Chem. Int. Ed. 41 2596-9) which involves the Cu(I) —catalyzed dipolar cycloaddition reaction of a terminal alkyne and an azide. Initially, an intermediary ligand (IA or IB) can be used to append the appropriate functional group (either an alkyne or azide) on to an agarose bead functionalized with free amino groups (Pumma, S., et al. (2005) Bioconjugate Chem. 38:1536-41). Either an azide (Scheme 5a) or an alkyne (Scheme 5b) functional group can be incorporated on the agarose bead. Subsequent contact of the newly functionalized bead with an appropriately substituted D609 or D609-like molecule (Scheme 6) would yield the affinity column matrix. Another example for coupling the agarose bead to a D609 or D609-like molecule is shown in Scheme 5c, which utilizes the coupling of an agarose bead functionalized with epoxide (Tosoh Bioscience) with a hydroxyl substituted D609 or D609-like molecule under basic conditions. In addition to functionalized agarose beads, pre-activated silica gels can be used as the synthesis of azido-modified silica gel for use in click chemistry to append a alkyne functionalized molecule has been reported (Linder, et al. (2006) Tetrahedron Lett. 47: 8721).

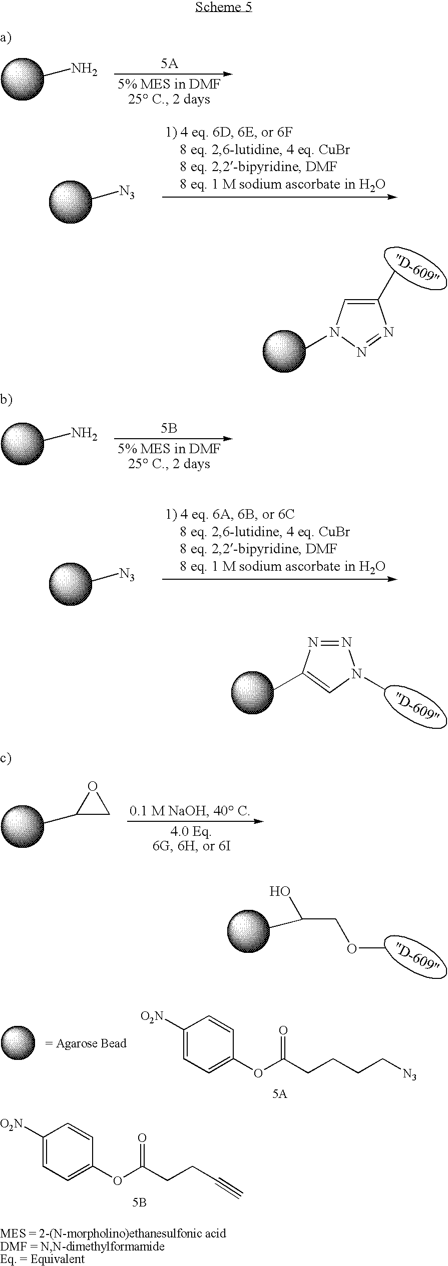

Wherein reagents 6A-I are of the formulas shown in Scheme 6 below.

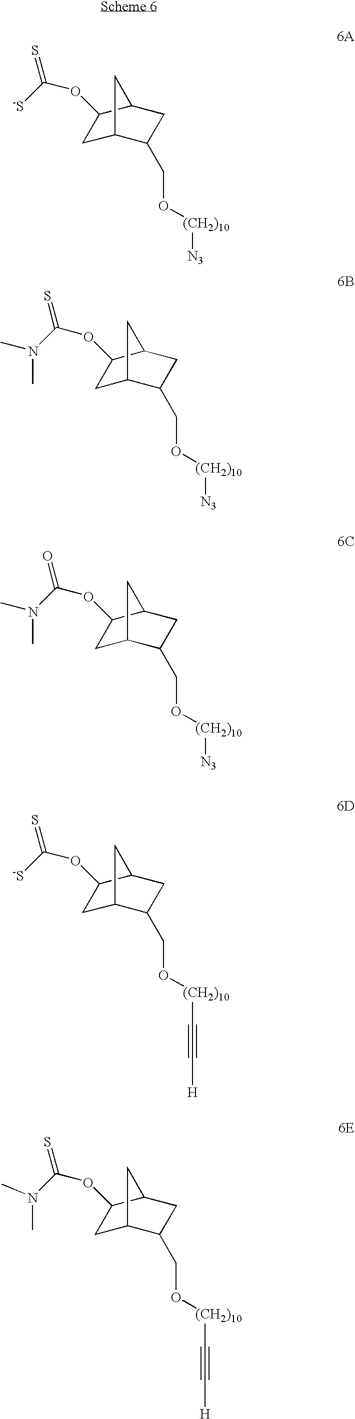

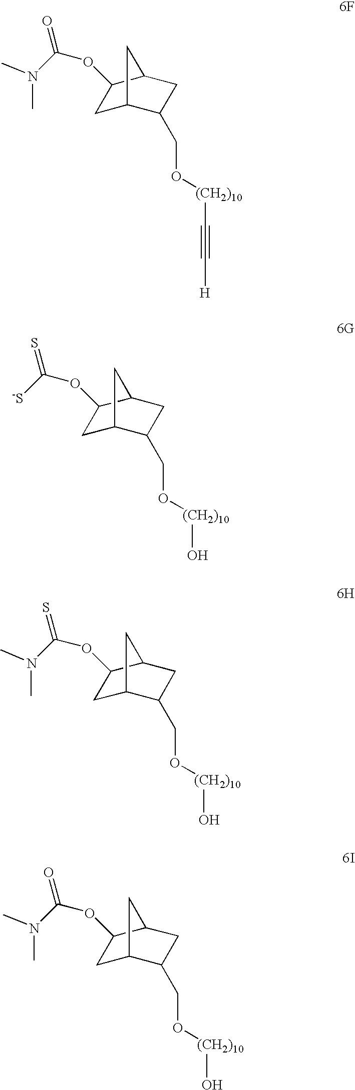

### Example 5

Analogous to the examples provided above, other moieties can be appended to the desired norbornane derivative or D-609 like derivative (to result in X and Y groups in the compounds of the invention) using an appropriate solvent, as shown in Schemes 7 and 8 below.

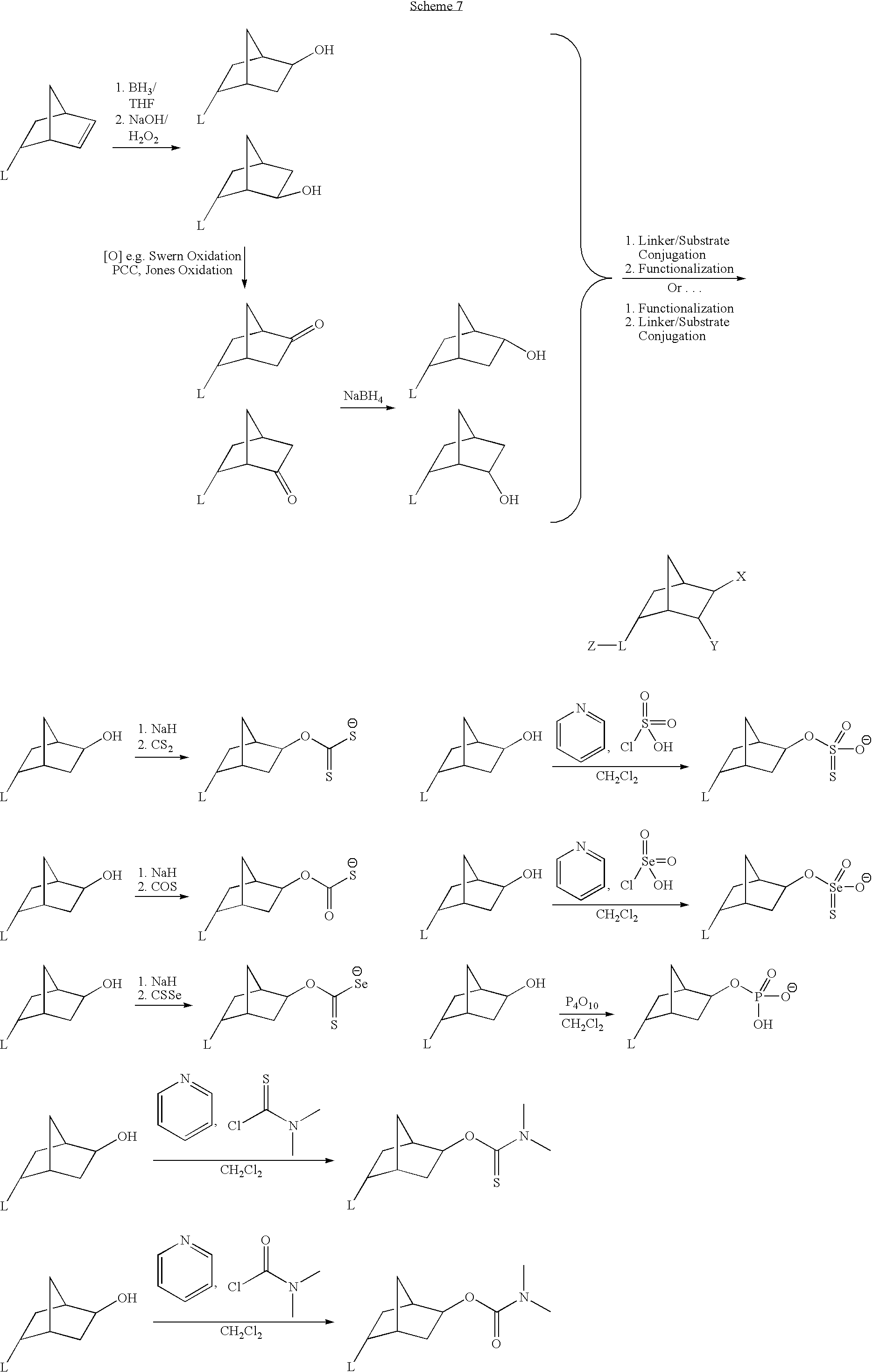

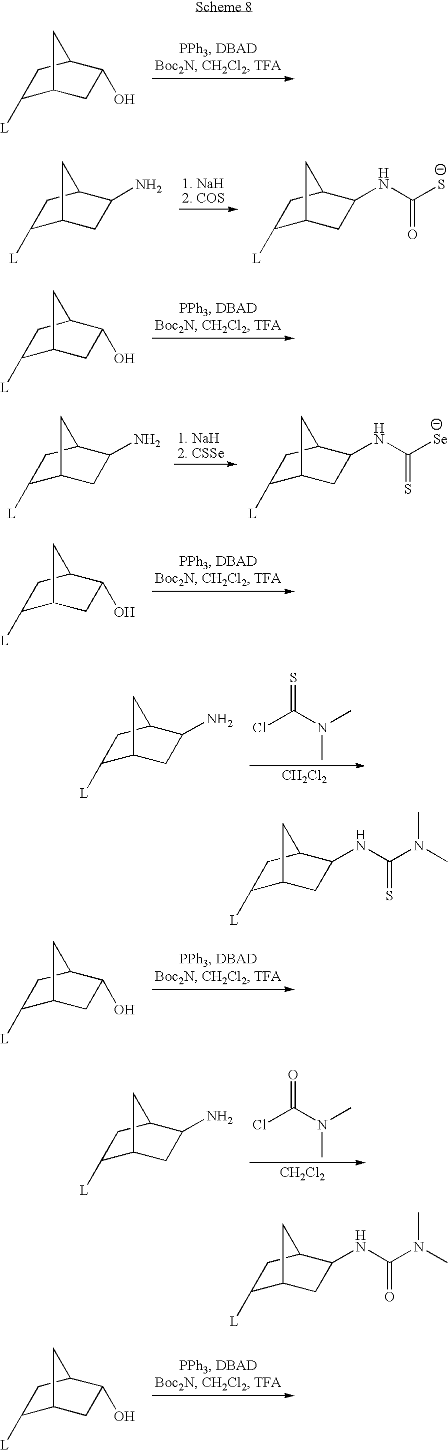

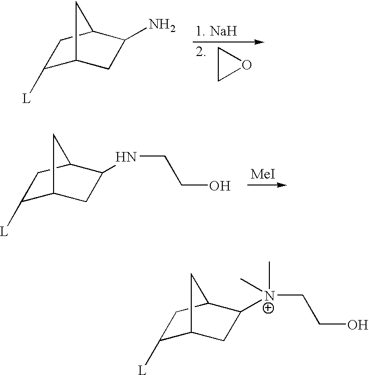

### Use of Complexes to Purify PC-PLC

Any biological sample suspected of containing PC-PLC is a suitable sample. Such samples include, but are not limited to rat alveolar macrophage cell line NR8383, human monocyte cell lines, and human and other mammalian lung. Suitable cells or tissue can be purchased from a commercial vendor or alternatively, one of skill in the art can utilize cells or tissue directly isolated from biological specimens. Cells and/or tissues are disrupted by addition of a non-ionic detergent in phosphate buffered saline (PBS) which are then contacted with a solid support containing the composition of this invention.

A cell sample may be tested for containing PC-PLC by a known assay, such as the Amplex Red Phosphatidylcholine-specific phospholipase C Assay Kit (A-12218, sold by Invitrogen, Carlsbad, Calif.). This assay provides methods for continuous in vitro monitoring of phospholipase activity in cell extracts or for inhibitor screening. The assay is based on a peroxidase-linked detection scheme and are suitable for use with either a fluorescence microplate reader or a fluorometer that has a time-base readout mode. Phosphorylcholine produced by PC-PLC action on phosphatidylcholine is hydrolyzed by alkaline phosphatase to generate choline. Experiments with purified PC-PLC from Bacillus cereus indicate that the Amplex Red PC-PLC Assay Kit can detect PC-PLC levels as low as 0.2 mU/mL using a reaction time of one hour. One Unit is defined as the amount of enzyme that will liberate 1 millimole of water-soluble organic phosphorus from L-a-phosphatidylcholine per minute at pH 7.3 at 37° C. This assay is further discussed in Moreno-Garcia, et al. “CD38 Signaling Regulates B Lymphocyte Activiation via a Phospholipase C(PLC)-γ2-Independent, Protein Kinase C, Phosphatidylcholine-PLC, and Phospholipase D-Dependent Signaling Cascade” J. Immun. 2005, 174:2867-2695, which is hereby incorporated by reference.

In one aspect, the solid support is a bead and accordingly, the modified resin is in a column and the cell and/or tissue homogenate is loaded onto the column. Proteins that do not bind to the complex of this invention are eluted with sodium phosphate buffer (optionally isotonic), having a pH of about 7.0. Dissociation of the protein from the complex is accomplished by lowering the buffer pH to about 6.0 or gradually increasing the ionic strength of the buffer until about 5 M with a sodium chloride solution. If necessary, elution can be done with buffer containing D609 followed by dialysis to remove the D609 from the eluted protein.

Once the protein is eluted a variety of characterizations may be performed. For example, the peptide/protein sequencing is done after the protein is purified such as via high performance liquid chromatography (“HPLC”). In one embodiment, the isolated purified protein is subjected to partial chemical or enzymatic cleavage to generate peptide fragments. These peptides are then separated by HPLC (narrow bore) and then analyzed by mass spectrometry. The different isolated sequences are used to generate one or more oligonucleotide probes. The data obtained by mass spectrometry using available program and databases will determine the DNA sequence from the predicted tryptic peptide sequences. The determined sequence can then be compared to genome databases to determine whether this gene has been previously identified as a protein with a function different from PC-PLC or only as a suspected protein. Primer pairs may be designed and synthesized to produce a cDNA (or cDNAs if more than one protein is isolated) using PCR. The eDNA(s) may be cloned into pGEX-6P to obtain a GST fusion protein that can be purified using glutathione-agarose beads.

In one aspect of the invention, there is provided a method of isolating eukaryotic PC-PLC comprising:
a. contacting a cell homogenate comprising eukaryotic PC-PLC with a compound provided herein, under conditions to form a complex between the compound and PC-PLC;
b. releasing mammalian PC-PLC from the complex, thereby isolating said PC-PLC.

In some embodiments, the cell homogenate is assayed to determined presence of PC-PLC prior to the contacting step.

In some embodiments, the releasing of PC-PLC from the complex is by adjusting the conditions to release the PC-PLC from the complex.

In some embodiments, conditions are affinity chromatography conditions.

In some embodiments, the conditions comprise a buffered solution.

In some embodiments, conditions comprise varying ionic strength of the solution.

In some embodiments, the solution has a pH of from about 6.5 to about 7.5.

In some embodiments, the solution has pH of about 7.0.

In some embodiments, the conditions that release PC-PLC from the complex comprises reducing the pH by at least 0.5.

In some embodiments, the method comprises the step of recovering uncomplexed PC-PLC before releasing PC-PLC from the complex.

In some embodiments, the method further comprises
c. purifying the PC-PLC by high pressure liquid chromatography to provide substantially pure PC-PLC.

In some embodiments, the method further comprises:
c. determining the amino acid sequence of isolated PC-PLC.

In some embodiments, the method further comprises:
d. determining polynucleotide sequence encoding mammalian PC-PLC.

In some embodiments, the method further comprises:
c. characterizing the isolated PC-PLC by mass spectrometry.

In some embodiments, the PC-PLC is mammalian PC-PLC.

In some embodiments, the method the PC-PLC is human PC-PLC.

The preceding discussion and examples are intended merely to illustrate the art. As is apparent to one of skill in the art, various modifications can be made to the above without departing from the spirit and scope of this invention.

What is claimed is:

1. A compound of formula I or II: 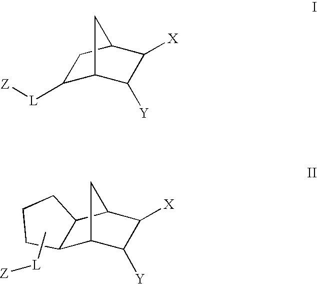
wherein;
either X or Y is selected from the group consisting of 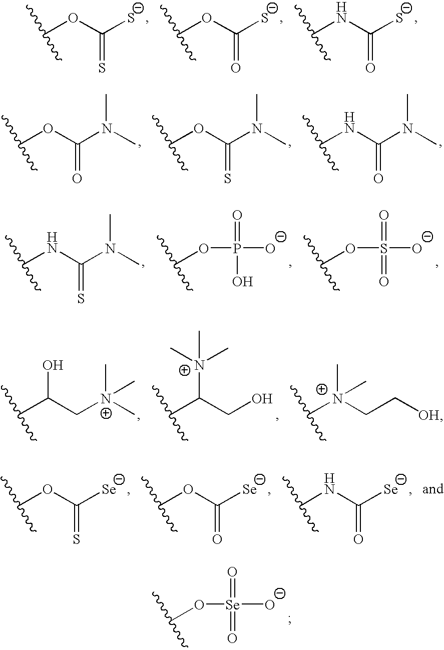
the other of X or Y is hydrogen;
L is a linker; and
Z is a solid support,
or a stereoisomer or salt thereof.

2. The compound of claim 1, wherein X or Y is selected from the group consisting of

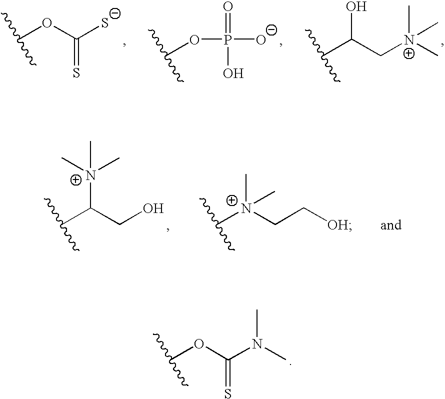

3. The compound of claim 1, wherein Z is a solid support selected from the group consisting of polystyrene, polystyrene resin grafted with polyethylene glycol, polyamide resin, polyacrylamide resin, polydimethylacrylamide resin, silica, dextran, agarose, and a cross-linked agarose.

4. The compound of claim 1, wherein L is selected from the group consisting of —(CH₂)ₘ—, —(CRR¹)ₘ—, —(CRR¹)ₘ—NR—, —(CRR¹)ₘ—O—, —(CRR¹)ₘ—O—(CRR¹)ₘ—O—, —(CRR¹)ₘ—S—, —(CRR¹)ₘ—C(O)—, —(CRR¹)ₘ—CO₂—, —(CRR¹)ₘ—S₂—, —(CRR¹)ₘ—OP(O)(OH)O—, —(CRR¹)ₘ—C(O)—NR—, —(CRR¹)ₘ—NRC(O)—, —(CRR¹)ₘ—NRC(S)—, —(CRR¹)ₘ—OC(O)—, —(CRR¹)ₘ—SO₂NR—, —(CRR¹)ₘ—NRC(O)NR—, —(CRR¹)ₘ—NRC(S)NR—, —(CRR¹)ₘ—OC(O)NR—, —(CRR¹)ₘ—O—(CRR¹)ₘ-heteroaryl, and —(CRR¹)ₘ—O—(CRR¹)ₘ—O—CH(OH)—; each of R and R¹ are independently selected from the group consisting of H, alkyl and heteroaryl; and m is 0-20.

5. The compound of claim 1, wherein L is —(CH₂)ₘ—, —(CH₂)ₘ—O—, —(CRR¹)ₘ—O—(CRR¹)ₘ—O—, —(CRR¹)ₘ—O—(CRR¹)ₘ-heteroaryl, and —(CRR¹)ₘ—O—(CRR¹)ₘ—O—CH(OH)—; and m is 8-20;

6. A compound of formula I: 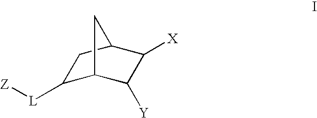
wherein; 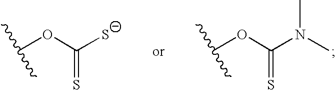
X is
Y is hydrogen;
L is —(CH₂)ₙ— or —(CH₂)ₙ—O—;
n is 8-20;
and Z is agarose;
or a stereoisomer or salt thereof.

7. A compound of formula III or IV: 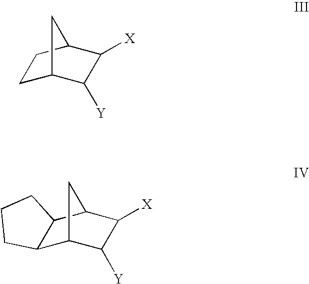
wherein either X and Y are selected from the group consisting of 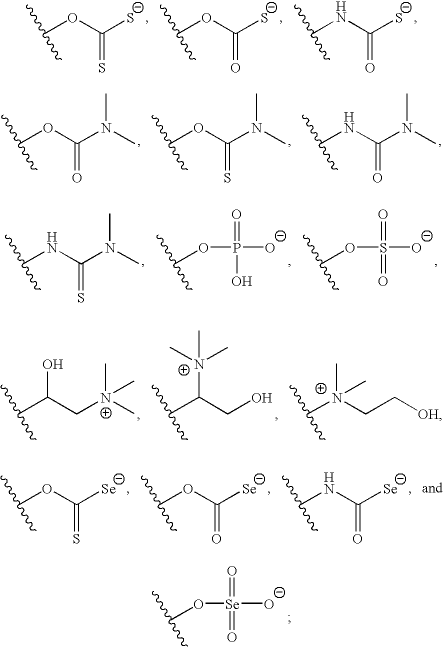
and
the other of X or Y is hydrogen,
or a stereoisomer or salt thereof,
with the proviso that when the compound is of the formula IV, X or Y is not

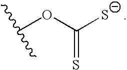

8. The compound of claim 1, wherein the compound is capable of complexing with eukaryotic PC-PLC.

9. A method of preparing a compound of formula I: 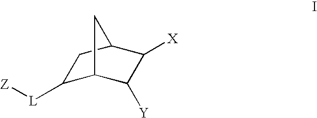
wherein;
either X or Y is selected from the group consisting of 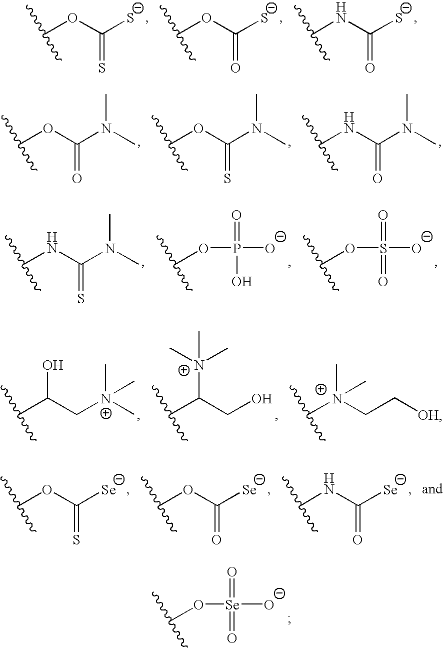
the other of X or Y is hydrogen;
L is a linker; and
Z is a solid support,
or a stereoisomer or salt thereof,
comprising contacting a compound of formula V or VI 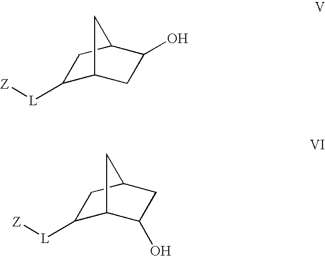
under reaction conditions to form the compound of formula I.

10. The method of claim 9, further comprising isolating the compound of formula I.

11. The method of claim 9, wherein the compound of formula V or VI: 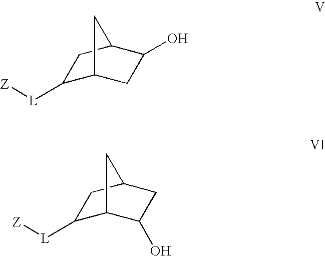
wherein L is a linker;
Z is a solid support,
or a stereoisomer or salt thereof,
is prepared by contacting under tethering conditions, a compound of formula VII or VIII, 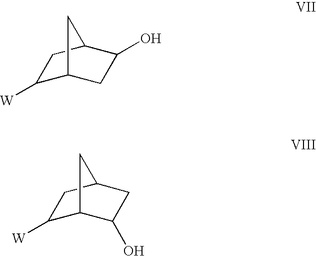
wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20.

12. A method of preparing a compound of formula I: 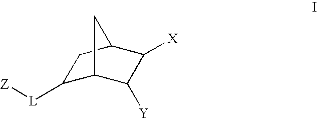
wherein either X or Y is selected from the group consisting of 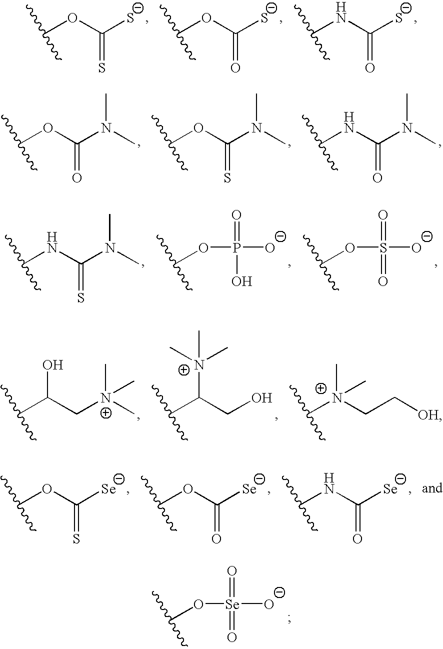
the other of X or Y is hydrogen;
L is a linker;
Z is a solid support,
or a stereoisomer or salt thereof,
is prepared by contacting under tethering conditions, a compound of formula IX 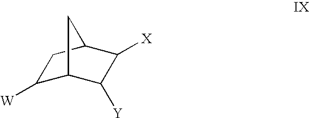
wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
under reaction conditions to form the compound of formula I.

13. A method of preparing a compound of formula IX: 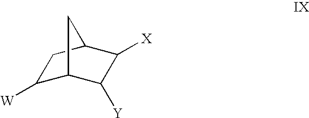
wherein;
either X or Y is selected from the group consisting of 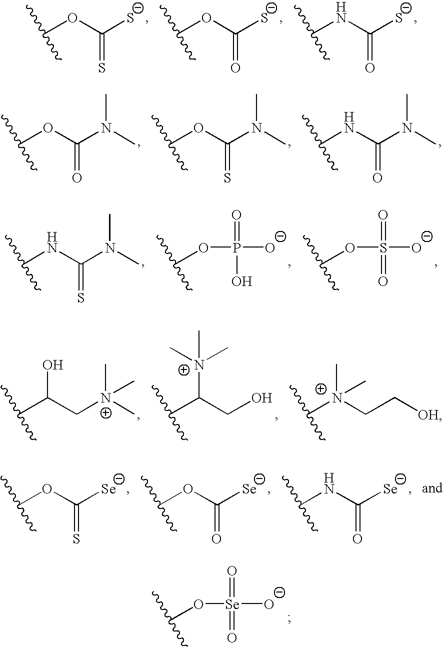
the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof,
comprising contacting a compound of formula VII or VIII 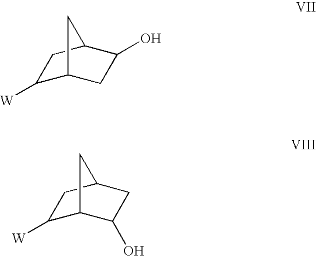
under reaction conditions to form the compound of formula I.

14. A method of preparing a compound of formula II: 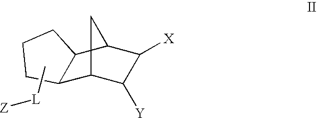
wherein;
either X or Y is selected from the group consisting of 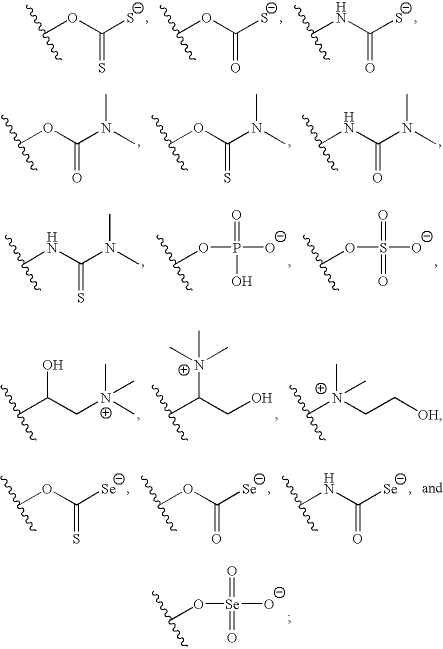
the other of X or Y is hydrogen;
L is a linker; and
Z is a solid support,
or a stereoisomer or a salt thereof,
comprising contacting a compound of formula X or XI 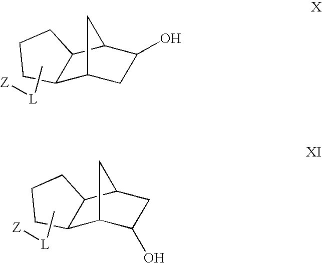
under reaction conditions to form the compound of formula II.

15. The method of claim 14, wherein the compound of formula X or XI: 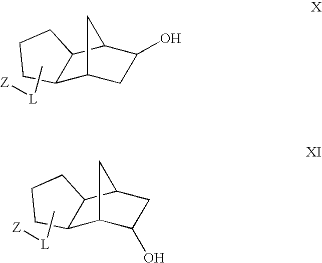
is prepared by contacting under tethering conditions, a compound of formula XII or XIII, 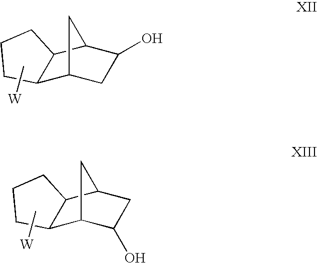
wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20.

16. A method of preparing a compound of formula II: 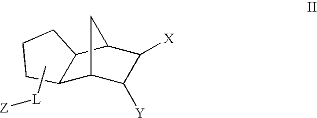
wherein either X or Y is selected from the group consisting of 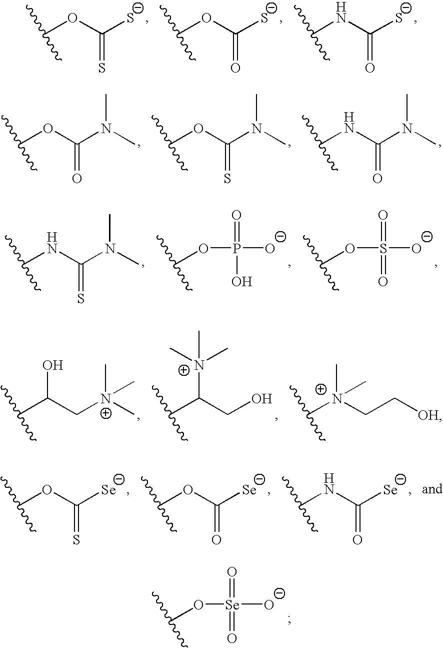
the other of X or Y is hydrogen;
L is a linker;
Z is a solid support,
or a stereoisomer or salt thereof,
is prepared by contacting under tethering conditions, a compound of formula XIV 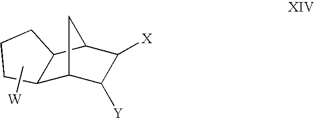
wherein;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
under reaction conditions to form the compound of formula I.

17. A method of preparing a compound of formula XIV: 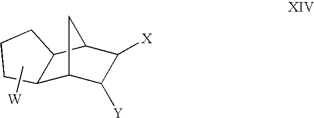
wherein;
either X or Y is selected from the group consisting of 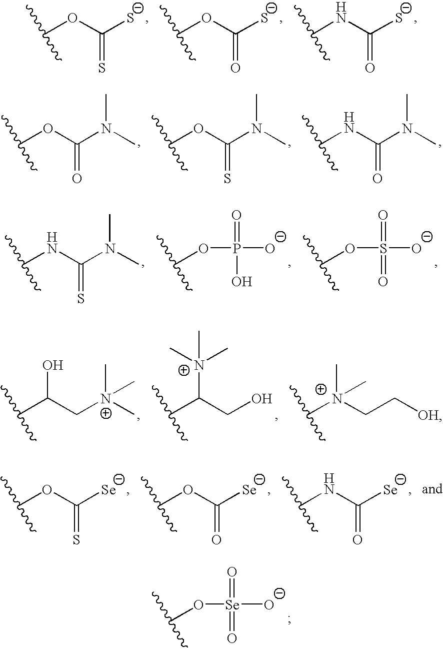
the other of X or Y is hydrogen;
W is selected from the group consisting of —(CRR¹)ₘ—X, —(CRR¹)ₘ—NH₂, —(CRR¹)ₘ—NHR, —(CRR¹)ₘ—SH, —(CRR¹)ₘ—OH, —(CRR¹)ₘ—O—(CRR¹)ₘ—OH, —(CRR¹)ₘ—C(O)H, —(CRR¹)ₘ—CO₂X, —(CRR¹)ₘ—S₂H, —(CRR¹)ₘ—OP(O)(OH)₂, —(CRR¹)ₘ—C(O)—NHR, —(CRR¹)ₘ—NRC(O)X, —(CRR¹)ₘ—NRC(S)X, —(CRR¹)ₘ—OC(O)X, —(CRR¹)ₘ—SO₂NHR, —(CRR¹)ₘ—NRC(O)NHR, —(CRR¹)ₘ—NRC(S)NHR, —(CRR¹)ₘ—OC(O)NHR, —(CRR¹)ₘ—N₃, —(CRR¹)ₘ—O—(CRR¹)ₘ—N₃, —(CRR¹)ₘ—C≡CH, and —(CRR¹)ₘ—O—(CRR¹)ₘ—C≡CH;
R and R¹ are independently selected from the group consisting of hydrogen, halo, hydroxyl, alkyl, alkoxy, and heteroaryl;
X is halo or alkoxy; and
m is 0-20,
or a stereoisomer or salt thereof,
comprising contacting a compound of formula XII or XIII 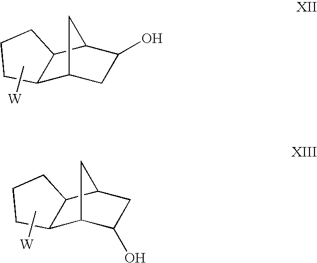
under reaction conditions to form the compound of formula I.

18. A method of isolating eukaryotic PC-PLC comprising:
a. contacting a cell homogenate comprising eukaryotic PC-PLC with a compound of claim 1 under conditions to form a complex between the compound and PC-PLC;
b. releasing mammalian PC-PLC from the complex, thereby isolating said PC-PLC.

19. The method of claim 18, wherein the PC-PLC is mammalian PC-PLC.

20. The method of claim 18, wherein the PC-PLC is human PC-PLC.
